SEQUENCE LISTING (42 sequences)

SEQ ID NO: 1 - protein, 625 aa, Homo sapiens [sequence omitted]

SEQ ID NO: 2 - DNA, 3278 bp, Homo sapiens
  source 1..3278: mol_type = genomic DNA
aggcacacaggcaaaatcaagttctacatctgtccctgtgtatgtcacttgtttgaatacgaaataaaattaaaaaaataaattcagtgtattgagaaagcaagcaattctctcaaggtatatttctgacatactaagattttaacgactttcacaaatatgctgtactgagagagaatgttacataacattgagaactagtacaagtaaatattaaagtgaagtgaccatttcctacacaagctcattcagaggaggatgaagaccattttggaggaagaaaagcacccttattaagaattgcagcaagtaagccaacaaggtcttttcaggatgattttcttatatcaagtggtacatttcattttatttacttcagtttctggtgaatgtgtgactcagttgttgaaggacacctgctttgaaggaggggacattactacggtcttcacaccaagcgccaagtactgccaggtagtctgcacttaccacccaagatgtttactcttcactttcacggcggaatcaccatctgaggatcccacccgatggtttacttgtgtcctgaaagacagtgttacagaaacactgccaagagtgaataggacagcagcgatttctgggtattctttcaagcaatgctcacaccaaataagcgcttgcaacaaagacatttatgtggacctagacatgaagggcataaactataacagctcagttgccaagagtgctcaagaatgccaagaaagatgcacggatgacgtccactgccactttttcacgtacgccacaaggcagtttcccagcctggagcatcgtaacatttgtctactgaagcacacccaaacagggacaccaaccagaataacgaagctcgataaagtggtgtctggattttcactgaaatcctgtgcactttctaatctggcttgtattagggacattttccctaatacggtgtttgcagacagcaacatcgacagtgtcatggctcccgatgcttttgtctgtggccgaatctgcactcatcatcccggttgcttgttttttaccttcttttcccaggaatggcccaaagaatctcaaagaaatctttgtctccttaaaacatctgagagtggattgcccagtacacgcattaaaaagagcaaagctctttctggtttcagtctacaaagctgcaggcacagcatcccagtgttctgccattcttcattttaccatgacactgatttcttgggagaagaactggatattgttgctgcaaaaagtcacgaggcctgccagaaactgtgcaccaatgccgtccgctgccagttttttacctataccccagcccaagcatcctgcaacgaagggaagggcaagtgttacttaaagctttcttcaaacggatctccaactaaaatacttcacgggagaggaggcatctctggatacacattaaggttgtgtaaaatggataatgagtgtaccaccaaaatcaagcccaggatcgttggaggaactgcgtctgttcgtggtgagtggccgtggcaggtgaccctgcacacaacctcacccactcagagacacctgtgtggaggctccatcattggaaaccagtggatattaacagccgctcactgtttctatggggtagagtcacctaagattttgcgtgtctacagtggcattttaaatcaatctgaaataaaagaggacacatctttctttggggttcaagaaataataatccatgatcagtataaaatggcagaaagcgggtatgatattgccttgttgaaactggaaaccacagtgaattacacagattctcaacgacccatatgcctgccttccaaaggagatagaaatgtaatatacactgattgctgggtgactggatgggggtacagaaaactaagagacaaaatacaaaatactctccagaaagccaagatacccttagtgaccaacgaagagtgccagaagagatacagaggacataaaataacccataagatgatctgtgccggctacagggaaggagggaaggacgcttgcaagggagattcgggaggccctctgtcctgcaaacacaatgaggtctggcatctggtaggcatcacgagctggggcgaaggctgtgctcaaagggagcggccaggtgtttacaccaacgtggtcgagtacgtggactggattctggagaaaactcaagcagtgtgaatgggttcccaggggccattggagtccctgaaggacccaggatttgctgggagagggtgttgagttcactgtgccagcatgcttcctccacagtaacacgctgaaggggcttggtgtttgtaagaaaatgctagaagaaaacaaactgtcacaagttgttatgtccaaaactcccgttctatgatcgttgtagtttgtttgagcattcagtctctttgtttttgatcacgcttctatggagtccaagaattaccataaggcaatatttctgaagattactatataggcagatatagcagaaaataaccaagtagtggcagtggggatcaggcagaagaactggtaaaagaagccaccataaatagatttgttcgatgaaagatgaaaactggaagaaaggagaacaaagacagtcttcaccattttgcaggaatctacactctgcctatgtgaacacatttcttttgtaaagaaagaaattgattgcatttaatggcagattttcagaatagtcaggaattcttgtcatttccattttaaaatatatattaaaaaaaatcagttcgagtagacacgagctaagagtgaatgtgaagataacagaatttctgtgtggaagaggattacaagcagcaatttacctggaagtgataccttaggggcaatcttgaagatacactttcctgaaaaatgatttgtgatggattgtatatttatttaaaatatcttgggaggggaggctgatggagatagggagcatgctcaaacctccctaagacaagctgctgctgtgactatgggctcccaaagagctagatcgtatatttatttgacaaaaatcaccatagactgcatccatactacagagaaaaaacaattagggcgcaaatggatagttacagtaaagtcttcagcaagcagctgcctgtattctaagcactgggattttctgtttcgtgcaaatatttatctcattattgttgtgatctagttcaataacctagaatttgaattgtcaccacatagctttcaatctgtgccaacaactatacaattcatcaagtgtg

SEQ ID NO: 3 - protein, 5 aa, synthetic construct
TAAMS

SEQ ID NO: 4 - protein, 17 aa, synthetic construct
GISGSGSSTYYADSVKG

SEQ ID NO: 5 - protein, 13 aa, synthetic construct
ELSYLYSGYYFDY

SEQ ID NO: 6 - protein, 7 aa, synthetic construct
GFTFSTA

SEQ ID NO: 7 - protein, 6 aa, synthetic construct
SGSGSS

SEQ ID NO: 8 - protein, 13 aa, synthetic construct
ELSYLYSGYYFDY

SEQ ID NO: 9 - protein, 122 aa, synthetic construct
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSS

SEQ ID NO: 10 - DNA, 366 bp, synthetic construct
  source 1..366: mol_type = other DNA
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccggtatctctggttctggttcttctacctactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctca

SEQ ID NO: 11 - protein, 452 aa, synthetic construct
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1356 bp, synthetic construct
  source 1..1356: mol_type = other DNA
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccggtatctctggttctggttcttctacctactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 13 - protein, 13 aa, synthetic construct
SGSSSNIGSNDVS

SEQ ID NO: 14 - protein, 7 aa, synthetic construct
KNYNRPS

SEQ ID NO: 15 - protein, 11 aa, synthetic construct
SAWDQRQFDVV

SEQ ID NO: 16 - protein, 9 aa, synthetic construct
SSSNIGSND

SEQ ID NO: 17 - skipped

SEQ ID NO: 18 - protein, 8 aa, synthetic construct
WDQRQFDV

SEQ ID NO: 19 - protein, 110 aa, synthetic construct
DIVLTQPPSVSGAPGQRVTISCSGSSSNIGSNDVSWYQQLPGTAPKLLIYKNYNRPSGVPDRFSGSKSGTSASLAITGLQAEDEADYYCSAWDQRQFDVVFGGGTKLTVL

SEQ ID NO: 20 - DNA, 330 bp, synthetic construct
  source 1..330: mol_type = other DNA
gatatcgtgctgacccagccgccgagcgtgagcggtgcaccgggccagcgcgtgaccattagctgtagcggcagcagcagcaacattggttctaacgacgtgtcttggtaccagcagctgccgggcacggcgccgaaactgctgatctacaaaaactacaaccgcccgagcggcgtgccggatcgctttagcggatccaaaagcggcaccagcgccagcctggcgattaccggcctgcaagcagaagacgaagcggattattactgctctgcttgggaccagcgtcagttcgacgttgtgtttggcggcggcacgaagttaaccgtccta

SEQ ID NO: 21 - protein, 216 aa, synthetic construct
DIVLTQPPSVSGAPGQRVTISCSGSSSNIGSNDVSWYQQLPGTAPKLLIYKNYNRPSGVPDRFSGSKSGTSASLAITGLQAEDEADYYCSAWDQRQFDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 22 - DNA, 648 bp, synthetic construct
  source 1..648: mol_type = other DNA
gatatcgtgctgacccagccgccgagcgtgagcggtgcaccgggccagcgcgtgaccattagctgtagcggcagcagcagcaacattggttctaacgacgtgtcttggtaccagcagctgccgggcacggcgccgaaactgctgatctacaaaaactacaaccgcccgagcggcgtgccggatcgctttagcggatccaaaagcggcaccagcgccagcctggcgattaccggcctgcaagcagaagacgaagcggattattactgctctgcttgggaccagcgtcagttcgacgttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 23 - protein, 5 aa, synthetic construct
TAAMS

SEQ ID NO: 24 - protein, 17 aa, synthetic construct
GISGSGSSTYYADSVKG

SEQ ID NO: 25 - protein, 13 aa, synthetic construct
ELSYLYSGYYFDY

SEQ ID NO: 26 - protein, 7 aa, synthetic construct
GFTFSTA

SEQ ID NO: 27 - protein, 6 aa, synthetic construct
SGSGSS

SEQ ID NO: 28 - protein, 13 aa, synthetic construct
ELSYLYSGYYFDY

SEQ ID NO: 29 - protein, 122 aa, synthetic construct; identical to SEQ ID NO: 9

SEQ ID NO: 30 - DNA, 366 bp, synthetic construct
  source 1..366: mol_type = other DNA
caggtgcagctgctggaatcaggcggcggactggtgcagcctggcggtagcctgagactgagctgcgctgctagtggcttcacctttagcaccgccgctatgagctgggttcgacaggccccagggaaaggcctcgagtgggtctcagggattagcggtagcggctctagcacctactacgccgatagcgtgaagggccggttcactatctctagggataactctaagaacaccctgtacctgcagatgaatagcctgagagccgaggacaccgccgtctactactgcgctagagagctgagctacctgtatagcggctactacttcgactactggggtcaaggcaccctggtcaccgtgtctagc

SEQ ID NO: 31 - protein, 452 aa, synthetic construct
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 1356 bp, synthetic construct
  source 1..1356: mol_type = other DNA
caggtgcagctgctggaatcaggcggcggactggtgcagcctggcggtagcctgagactgagctgcgctgctagtggcttcacctttagcaccgccgctatgagctgggttcgacaggccccagggaaaggcctcgagtgggtctcagggattagcggtagcggctctagcacctactacgccgatagcgtgaagggccggttcactatctctagggataactctaagaacaccctgtacctgcagatgaatagcctgagagccgaggacaccgccgtctactactgcgctagagagctgagctacctgtatagcggctactacttcgactactggggtcaaggcaccctggtcaccgtgtctagcgctagcactaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaactgctgggcggcccttctgtgttcctgttccctccaaagcccaaggacaccctgatgatctcccggacccctgaagtgacctgcgtggtggtggccgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctggccgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 33 - protein, 13 aa, synthetic construct
SGSSSNIGSNDVS

SEQ ID NO: 34 - protein, 7 aa, synthetic construct
KNYNRPS

SEQ ID NO: 35 - protein, 11 aa, synthetic construct
SAWDQRQFDVV

SEQ ID NO: 36 - protein, 9 aa, synthetic construct
SSSNIGSND

SEQ ID NO: 37 - skipped

SEQ ID NO: 38 - protein, 8 aa, synthetic construct
WDQRQFDV

SEQ ID NO: 39 - protein, 110 aa, synthetic construct
QSVLTQPPSASGTPGQRVTISCSGSSSNIGSNDVSWYQQLPGTAPKLLIYKNYNRPSGVPDRFSGSKSGTSASLAISGLQSEDEADYYCSAWDQRQFDVVFGGGTKLTVL

SEQ ID NO: 40 - DNA, 330 bp, synthetic construct
  source 1..330: mol_type = other DNA
cagtcagtcctgactcagccccctagcgctagtggcacccctggtcaaagagtgactattagctgtagcggctctagctctaatatcggctctaacgacgtcagctggtatcagcagctgcccggcaccgcccctaagctgctgatctataagaactataataggcctagcggcgtgcccgataggtttagcggatctaaatcagggacttctgctagtctggctattagcggcctgcagtcagaggacgaggccgactactactgtagcgcctgggatcagcgtcagttcgacgtggtgttcggcggaggcactaagctgaccgtgctg

SEQ ID NO: 41 - protein, 216 aa, synthetic construct
QSVLTQPPSASGTPGQRVTISCSGSSSNIGSNDVSWYQQLPGTAPKLLIYKNYNRPSGVPDRFSGSKSGTSASLAISGLQSEDEADYYCSAWDQRQFDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - DNA, 648 bp, synthetic construct
  source 1..648: mol_type = other DNA
cagtcagtcctgactcagccccctagcgctagtggcacccctggtcaaagagtgactattagctgtagcggctctagctctaatatcggctctaacgacgtcagctggtatcagcagctgcccggcaccgcccctaagctgctgatctataagaactataataggcctagcggcgtgcccgataggtttagcggatctaaatcagggacttctgctagtctggctattagcggcctgcagtcagaggacgaggccgactactactgtagcgcctgggatcagcgtcagttcgacgtggtgttcggcggaggcactaagctgaccgtgctgggtcaacctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

(19) United States
(12) Patent Application Publication
(10) Pub. No.: US 2024/0077497 A1
(43) Pub. Date: Mar. 7, 2024

(54) METHODS FOR THE DETECTION OF ANTI-DRUG ANTIBODIES AGAINST FACTOR XI AND/OR FACTOR XIA ANTIBODIES

(71) Applicant: Anthos Therapeutics, Inc., Cambridge, MA (US)

(72) Inventors: Debra A. FREEDHOLM, Cambridge, MA (US); Daniel M. BLOOMFIELD, Cambridge, MA (US); Royston J. GLASSPOOL, Cambridge, MA (US); Jonathan E. FREEMAN, Cambridge, MA (US); Yasser KHDER, Rosenau (FR)

(21) Appl. No.: 18/335,507

(22) Filed: Jun. 15, 2023

Related U.S. Application Data
(63) Continuation of application No. PCT/US21/64117, filed on Dec. 17, 2021.
(60) Provisional application No. 63/127,536, filed on Dec. 18, 2020.

(51) Int. Cl. G01N 33/68; G01N 33/86

(52) U.S. Cl. CPC G01N 33/686; G01N 33/86; G01N 2333/96452; G01N 2800/52

(57) ABSTRACT

This disclosure relates to methods for detection and measurement of anti-drug antibodies (ADAs) against Factor XI and/or Factor XIa therapeutic antibodies, e.g., in a subject being treated with said Factor XI and/or Factor XIa therapeutic antibodies.

### CROSS-REFERENCE TO RELATED APPLICATIONS

This application is a continuation of International (PCT) Patent Application No. PCT/US2021/064117, filed on Dec. 17, 2021, which claims the benefit of and priority to U.S. Provisional Patent Application No. 63/127,536, filed on Dec. 18, 2020, the disclosures of each of which are hereby incorporated by reference in their entireties for all purposes.

### SEQUENCE LISTING

The instant application contains a Sequence Listing which has been submitted electronically in XML format and is hereby incorporated by reference in its entirety. Said XML copy, created on Jun. 13, 2023, is named ATD-010WOC1_SL.xml and is 47,079 bytes in size.

### FIELD OF THE DISCLOSURE

The present disclosure relates generally to methods for detection and measurement of anti-drug antibodies (ADAs) against Factor XI and/or Factor XIa therapeutic antibodies, e.g., in a subject being treated with said Factor XI and/or Factor XIa therapeutic antibodies.

### BACKGROUND

There exists a high unmet medical need for safer therapies to reduce thromboembolic complications such as stroke, systemic embolism, cognitive decline and mortality, with comparable or improved efficacy to that exhibited by existing therapies, and with a lower risk of bleeding.

Factor XI (FXI) is a serine protease functioning both in the intrinsic and extrinsic coagulation pathways. Factor XI exists in the zymogen form as a homodimer; upon cleavage of the peptide bond at R369-I370, Factor XI is activated (Factor XIa, FXIa). FXI plays a minor role in normal hemostasis in a high tissue factor environment but does play a key role in thrombosis. Genetic Factor XI deficiency is associated with decreased incidence of ischemic stroke and venous thromboembolic events (Salomon et al. 2008; Salomon, et al. (2011) Thromb Haemost.; 105:269-73). Bleeding manifestations in subjects with Factor XI deficiency are infrequent, often mild, result from injury or trauma, and very rarely affect critical organs (Salomon et al 2011).

Antibodies that bind Factor XI and/or Factor XIa have been studied. For example, WO 2016/207858 describes one such anti-Factor XI and/or Factor XIa antibody, disclosed in Table 1 of the present application as Antibody 1. The present disclosure adds to these developments and provides further clinical methods, including dosage regimens, to treat patients with specific thromboembolic disorders with desired safety and efficacy. Furthermore, the present disclosure adds to the earlier developments in the field by providing formulations comprising such FXI and/or FXIa antibodies that are sufficiently stable and suitable for administration to patients.

### SUMMARY OF THE DISCLOSURE

The present disclosure relates to methods for detection and measurement of anti-Factor XI and/or activated Factor XI (Factor XIa) antibodies, or antigen-binding fragments thereof.

Accordingly, in one aspect, provided herein is a method of detecting an anti-drug antibody (ADA) against an anti-Factor XI and/or anti-Factor XIa antibody or antigen-binding fragment thereof, wherein the method comprises: (a) incubating a sample with an acid to dissociate anti-Factor XI and/or anti-Factor XIa antibody-antigen complexes and/or dissociate anti-Factor XI and/or anti-Factor XIa antibody-ADA complexes present in the sample to create an acid digest, (b) incubating the acid digest on a plate coated with the anti-Factor XI and/or anti-Factor XIa antibody or antigen-binding fragment thereof, (c) neutralizing the acid digest, and (d) detecting the presence of the ADA using a ruthenylated detector cocktail.

In some embodiments, the sample is a sample from a subject. In some embodiments, the sample is selected from the group consisting of blood, plasma, or serum from a subject. In some embodiments, the method comprises an initial step of preparing the sample.

In some embodiments, the acid is selected from the group consisting of acetic acid, citric acid, phosphoric acid, and mixtures thereof. In some embodiments, the acid is acetic acid. In some embodiments, the acetic acid is at a concentration of about 300 mM.

In some embodiments, the antigen is Factor XI and/or Factor XIa. In some embodiments, the plate is coated with streptavidin. In some embodiments, the anti-Factor XI and/or anti-Factor XIa antibody or antigen-binding fragment thereof on the plate is at a concentration selected from the group consisting of about 0.1 μg/ml, about 0.25 μg/ml, about 0.5 μg/ml, about 0.75 μg/ml, and about 1 μg/ml. In some embodiments, the anti-Factor XI and/or anti-Factor XIa antibody or antigen-binding fragment thereof on the plate is at a concentration of about 0.25 μg/ml.

In some embodiments, the neutralizing allows for the ADA to bind the anti-Factor XI and/or anti-Factor XIa antibody or antigen-binding fragment thereof on the coated plate. In some embodiments, the neutralizing uses a base at a pH of about 8.0. In some embodiments, the neutralizing uses a base selected from the group consisting of Tris, Phosphate, HEPES, triethanolamine, and mixtures thereof. In some embodiments, the base is Tris.

In some embodiments, the ruthenylated detector cocktail comprises an antibody. In some embodiments, the antibody is selected from the group consisting of an anti-human IgG, an anti-human IgM, an anti-human IgE, an anti-rabbit Ig, and any combination thereof. In some embodiments, the detecting specifically detects the ADA and not Factor XI and/or Factor XIa.

In some embodiments, the method further comprises a washing after the incubating.

In some embodiments, the anti-Factor XI and/or anti-Factor XIa antibody or antigen-binding fragment thereof is at a concentration of about 0.25 μg/mL.

In some embodiments, the anti-Factor XI and/or anti-Factor XIa antibody or antigen-binding fragment thereof comprises a heavy chain variable region (VH) comprising complementary determining regions HCDR1, HCDR2, and HCDR3 in SEQ ID NO: 9 or 29; and a light chain variable region (VL) comprising complementary determining regions LCDR1, LCDR2, LCDR3 in SEQ ID NO: 19 or 39.

In some embodiments, the anti-Factor XI and/or anti-Factor XIa antibody or antigen-binding fragment thereof comprises: i. a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35; ii. a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of SEQ ID NO: 37; and a light chain variable region CDR3 of SEQ ID NO: 38; iii. a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of SEQ ID NO: 37; and a light chain variable region CDR3 of SEQ ID NO: 15; or iv. a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14; and a light chain variable region CDR3 of SEQ ID NO: 15.

In some embodiments, the anti-Factor XI and/or anti-Factor XIa antibody or antigen-binding fragment thereof comprises a heavy chain variable region (VH) selected from the group consisting of SEQ ID NO: 9, 29, and a VH with 90% identity thereto; and a light chain variable region (VL) selected from the group consisting of SEQ ID NO: 19, 39, and a VL with 90% identity thereto. In certain embodiments, the anti-Factor XI and/or anti-Factor XIa antibody or antigen-binding fragment thereof comprises a heavy chain variable region (VH) selected from the group consisting of SEQ ID NO: 9 and 29; and a light chain variable region (VL) selected from the group consisting of SEQ ID NO: 19 and 39.

In some embodiments, the anti-Factor XI and/or anti-Factor XIa antibody comprises a heavy chain comprising an amino acid sequence of SEQ ID NO: 31, 11, and a heavy chain with 90% identity thereto; and a light chain comprising an amino acid sequence of SEQ ID NO: 41, 21, and a light chain with 90% identity thereto. In certain embodiments, the anti-Factor XI and/or anti-Factor XIa antibody comprises a heavy chain comprising an amino acid sequence of SEQ ID NO: 31 and a light chain comprising an amino acid sequence of SEQ ID NO: 41.

In some embodiments, the anti-Factor XI and/or anti-Factor XIa antibody is a human monoclonal antibody. In some embodiments, the anti-Factor XI and/or anti-Factor XIa antibody is a human IgG1 isotype. In some embodiments, the anti-Factor XI and/or anti-Factor XIa antibody comprises D265A and P329A substitutions in the Fc domain.

In another aspect, provided herein is a method of detecting an anti-drug antibody (ADA) against an anti-Factor XI and/or anti-Factor XIa antibody or antigen-binding fragment thereof, wherein the antibody or antigen-binding fragment thereof comprises: i. a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35; ii. a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of SEQ ID NO: 37; and a light chain variable region CDR3 of SEQ ID NO: 38; iii. a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of SEQ ID NO: 37; and a light chain variable region CDR3 of SEQ ID NO: 15; or iv. a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14; and a light chain variable region CDR3 of SEQ ID NO: 15 and wherein the method comprises: (a) incubating a sample with an acid to dissociate anti-Factor XI and/or anti-Factor XIa antibody-antigen complexes and/or dissociate anti-Factor XI and/or anti-Factor XIa antibody-ADA complexes present in the sample to create an acid digest, (b) incubating the acid digest on a plate coated with the anti-Factor XI and/or anti-Factor XIa antibody or antigen-binding fragment thereof, (c) neutralizing the acid digest, and (d) detecting the presence of the ADA using a ruthenylated detector cocktail.

In some embodiments, the sample is a sample from a subject. In some embodiments, the sample is selected from the group consisting of blood, plasma, or serum from a subject. In some embodiments, the method comprises an initial step of preparing the sample.

In some embodiments, the acid is selected from the group consisting of acetic acid, citric acid, phosphoric acid, and mixtures thereof. In some embodiments, the acid is acetic acid. In some embodiments, the acetic acid is at a concentration of about 300 mM.

In some embodiments, the antigen is Factor XI and/or Factor XIa. In some embodiments, the plate is coated with streptavidin. In some embodiments, the anti-Factor XI and/or anti-Factor XIa antibody or antigen-binding fragment thereof on the plate is at a concentration selected from the group consisting of about 0.1 μg/ml, about 0.25 μg/ml, about 0.5 μg/ml, about 0.75 μg/ml, and about 1 μg/ml. In some embodiments, the anti-Factor XI and/or anti-Factor XIa antibody or antigen-binding fragment thereof on the plate is at a concentration of about 0.25 μg/ml.

In some embodiments, the neutralizing allows for the ADA to bind the anti-Factor XI and/or anti-Factor XIa antibody or antigen-binding fragment thereof on the coated plate. In some embodiments, the neutralizing uses a base at a pH of about 8.0. In some embodiments, the neutralizing uses a base selected from the group consisting of Tris, Phosphate, HEPES, triethanolamine, and mixtures thereof. In some embodiments, the base is Tris.

In some embodiments, the ruthenylated detector cocktail comprises an antibody. In some embodiments, the antibody is selected from the group consisting of an anti-human IgG, an anti-human IgM, an anti-human IgE, an anti-rabbit Ig, and any combination thereof. In some embodiments, the detecting specifically detects the ADA and not Factor XI and/or Factor XIa.

In some embodiments, the method further comprises a washing after the incubating.

In some embodiments, the anti-Factor XI and/or anti-Factor XIa antibody or antigen-binding fragment thereof is at a concentration of about 0.25 μg/mL.

In some embodiments, the anti-Factor XI and/or anti-Factor XIa antibody or antigen-binding fragment thereof comprises a heavy chain variable region (VH) selected from the group consisting of SEQ ID NO: 9, 29, and a VH with 90% identity thereto; and a light chain variable region (VL) selected from the group consisting of SEQ ID NO: 19, 39, and a VL with 90% identity thereto. In certain embodiments, the anti-Factor XI and/or anti-Factor XIa antibody or antigen-binding fragment thereof comprises a heavy chain variable region (VH) selected from the group consisting of SEQ ID NO: 9 and 29; and a light chain variable region (VL) selected from the group consisting of SEQ ID NO: 19 and 39.

In some embodiments, the anti-Factor XI and/or anti-Factor XIa antibody comprises a heavy chain comprising an amino acid sequence of SEQ ID NO: 31, 11, and a heavy chain with 90% identity thereto; and a light chain comprising an amino acid sequence of SEQ ID NO: 41, 21, and a light chain with 90% identity thereto. In certain embodiments, the anti-Factor XI and/or anti-Factor XIa antibody comprises a heavy chain comprising an amino acid sequence of SEQ ID NO: 31 and a light chain comprising an amino acid sequence of SEQ ID NO: 41.

In some embodiments, the anti-Factor XI and/or anti-Factor XIa antibody is a human monoclonal antibody. In some embodiments, the anti-Factor XI and/or anti-Factor XIa antibody is a human IgG1 isotype. In some embodiments, the anti-Factor XI and/or anti-Factor XIa antibody comprises D265A and P329A substitutions in the Fc domain.

Other embodiments and details of the disclosure are presented herein below.

### BRIEF DESCRIPTION OF THE DRAWINGS

FIG. 1 is a schematic depiction of the initial bridging assay for detection of anti-drug antibodies (ADA) in cynomolgus samples.

FIG. 2 is a schematic depiction of the modified bridging assay with a Factor XI/Factor XIa depletion step for detection of ADA in cynomolgus samples.

FIG. 3 is a schematic depiction of the modified bridging assay with a Ruthenylated anti-monkey Ig for detection of ADA in cynomolgus samples.

FIG. 4 is a schematic depiction of the ADA assay for cynomolgus samples.

FIG. 5 is a schematic depiction of the ADA assay for human samples.

### DETAILED DESCRIPTION

### Definitions

To facilitate an understanding of the present invention, a number of terms and phrases are defined below.

The terms “a” and “an” as used herein mean “one or more” and include the plural unless the context is inappropriate.

As used herein, the terms “FXI protein,” “FXI antigen,” and “FXI” are used interchangeably, and refers to the Factor XI protein in different species. Factor XI is the mammalian plasma coagulation factor XI, a glycoprotein present in human plasma at a concentration of 25-30 nM as a zymogen that when converted by limited proteolysis to an active serine protease, participates in the intrinsic pathway of blood coagulation.

The terms “FXIa protein,” “FXIa antigen,” and “FXIa”, are used interchangeably, and refers to the activated FXI protein in different species. The zymogen Factor XI is converted into its active form, the coagulation factor XIa (FXIa), either via the contact phase of blood coagulation or through thrombin-mediated activation on the platelet surface. During this activation of factor XI, an internal peptide bond is cleaved in each of the two chains, resulting in the activated factor XIa, a serine protease composed of two heavy and two light chains held together by disulfide bonds. This serine protease FXIa converts the coagulation Factor IX into IXa, which subsequently activates coagulation Factor X (Xa). Xa then can mediate coagulation Factor II/Thrombin activation. For example, human FXI has the sequence as set out in Table 1 (SEQ ID NO:1) and has been described in previous reports and literature (Mandle R J Jr, et al. (1979) Blood; 54(4):850; NCBI Reference Sequence: AAA51985).

In the context of this present disclosure, the terms “FXI” and “FXIa” (and the like) include mutants and variants of the natural FXI and FXIa protein, respectively, which have substantially the same amino acid sequence as that of the native primary structure (amino acid sequence) described in the above-mentioned reports.

The term “catalytic domain,” “serine protease catalytic domain,” and similar terms as used herein, means amino acids Ile370 to Val607, as counted from the Glu1 at the N-terminus of the mature protein that is in circulation. It can also be described as residues 388-625 at the C-terminus of FXI. As used herein, the term “active site” means the catalytic triad comprised of the amino acids His413, Asp462 and Ser557. (Bane and Gailani (2014) Drug Disc. 19(9)).

The term “antibody” as used herein means a whole antibody and any antigen binding fragment (i.e., “antigen-binding portion”) or single chain thereof. A whole antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. In some specific aspects, an antibody can be a monoclonal antibody, human antibody, humanized antibody, camelised antibody, or chimeric antibody. Antibodies can be of any isotype (e.g., IgG, IgE, IgM, IgD, IgA and IgY), class (e.g., IgG1, IgG2, IgG3, IgG4, IgA1 and IgA2) or subclass.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

As used herein, the terms “drug delivery formulation” or “intravenous drug delivery formulation” refers to a pharmaceutical formulation comprising the combination of an active agent with a carrier, inert or active, making the composition especially suitable for diagnostic or therapeutic use in vivo or ex vivo.

As used herein, the terms “subject” and “patient” refer to an organism to be treated by the methods and compositions described herein. Such organisms preferably include, but are not limited to, mammals (e.g., murines, simians, equines, bovines, porcines, primates, canines, felines, and the like), and more preferably include humans. In certain embodiments, the subject is a cynomolgus monkey. In certain embodiments, the subject is a human.

A “thromboembolic disorder,” or similar terms as used herein, refer to any number of conditions or diseases in which the intrinsic and/or common coagulation pathways are aberrantly activated or are not naturally deactivated (e.g., without therapeutic means). These conditions include but are not limited to thromboembolic stroke and other types of stroke of ischemic origin, atrial fibrillation, stroke prevention in atrial fibrillation (SPAF), deep vein thrombosis, venous thromboembolism, and pulmonary embolism. These can also include prevention and treatment of catheter-related thrombosis (e.g., Hickman catheter in oncology patients) in which catheters become thrombosed, and extracorporeal membrane oxygenation (ECMO), in which the tubing and oxygenation membrane develops clots.

A “thromboembolic disorder” or similar terms as used herein, can also refer to any number of the following, which the anti-FXI and/or FXIa antibodies or antigen binding fragments thereof of the present disclosure can be used to prevent or treat:
thromboembolism in subjects with suspected or confirmed cardiac arrhythmia such as paroxysmal, persistent or permanent atrial fibrillation or atrial flutter;
stroke prevention in atrial fibrillation (SPAF), a subpopulation of which is AF patients undergoing percutaneous coronary interventions (PCI);
acute venous thromboembolic events (VTE) treatment and extended secondary VTE prevention in patients at high risk for bleeding;
venous thromboembolism, wherein the subject is a pediatric subject (pediatric VTE);
cerebral and cardiovascular events in secondary prevention after transient ischemic attack (TIA) or non-disabling stroke and prevention of thromboembolic events in heart failure with sinus rhythm;
hemorrhagic stroke;
clot formation in left atrium and thromboembolism in subjects undergoing cardioversion for cardiac arrhythmia;
thrombosis before, during and after ablation procedure for cardiac arrhythmia;
venous thrombosis, this includes but not exclusively, treatment and secondary prevention of deep or superficial veins thrombosis in the lower members or upper member, thrombosis in the abdominal and thoracic veins, sinus thrombosis and thrombosis of jugular veins;
thrombosis on any artificial surface in the veins or arteries like catheter, pacemaker wires, synthetic arterial grafts; mechanical or biological heart valves or left ventricular assist device;
pulmonary embolism in patients with or without venous thrombosis;
Chronic Thromboembolic Pulmonary Hypertension (CTEPH);
arterial thrombosis on ruptured atherosclerotic plaque, thrombosis on intra-arterial prosthesis or catheter and thrombosis in apparently normal arteries, this includes but not limited to acute coronary syndromes, ST elevation myocardial infarction, non ST elevation myocardial infarction, unstable angina, stent thrombosis, thrombosis of any artificial surface in the arterial system and thrombosis of pulmonary arteries in subjects with or without pulmonary hypertension;
thrombosis and thromboembolism in patients undergoing percutaneous coronary interventions (PCI);
cardioembolic and cryptogenic strokes;
non-central nervous systemic embolism (non-CNS systemic embolism);
thrombosis in patients with invasive and non-invasive cancer malignancies;
thrombosis over an indwelling catheter;
thrombosis and thromboembolism in severely ill patients;
cardiac thrombosis and thromboembolism, including but not limited to cardiac thrombosis after myocardial infarction, cardiac thrombosis related to condition such as cardiac aneurysm, myocardial fibrosis, cardiac enlargement and insufficiency, myocarditis and artificial surface in the heart;
thromboembolism in patients with valvular heart disease with or without atrial fibrillation;
thromboembolism over valvular mechanic or biologic prostheses;
thromboembolism in patients who had native or artificial cardiac patches, arterial or venous conduit tubes after heart repair of simple or complex cardiac malformations;
venous thrombosis and thromboembolism after knee replacement surgery, hip replacement surgery, and orthopedic surgery, thoracic or abdominal surgery;
arterial or venous thrombosis after neurosurgery including intracranial and spinal cord interventions;
congenital or acquired thrombophilia including but not exclusively factor V Leiden, prothrombin mutation, antithrombin III, protein C and protein S deficiencies, factor XIII mutation, familial dysfibrinogenemia, congenital deficiency of plasminogen, increased levels of factor XI, sickle cell disease, antiphospholipid syndrome, autoimmune disease, chronic bowel disease, nephrotic syndrome, hemolytic uremia, myeloproliferative disease, disseminated intra vascular coagulation, paroxysmal nocturnal hemoglobinuria and heparin induced thrombopenia;
thrombosis and thromboembolism in chronic kidney disease; and
thrombosis and thromboembolism in patients undergoing hemodialysis and in patients undergoing extra-corporal membrane oxygenation.

As used herein, the term “trough” or “trough level” refers to the lowest concentration reached by a drug before the next dose of the drug is administered.

The terms “treat,” “treating,” or “treatment,” and other grammatical equivalents as used in this disclosure, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

As used herein, the term “vial” refers to a container that holds the drug product. In some embodiments, the vial may be a vial, a bag, a pen, or a syringe.

As used herein, the term “drug product” refers to an anti-Factor XI/XIa antibody described herein, e.g., Antibody 1 as disclosed in Table 1, and excipients, e.g., a histidine buffer, a sugar, and a polysorbate.

The term “about” refers to any minimal alteration in the concentration or amount of an agent that does not change the efficacy of the agent in preparation of a formulation and in treatment of a disease or disorder. In certain embodiments, the term “about” may include ±5%, ±10%, or ±15% of a specified numerical value or data point.

Ranges can be expressed in this disclosure as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed in this disclosure, and that each value is also disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

Anti-Factor XI and/or Activated Factor XI (Factor XIa) Antibodies

In some embodiments, the present disclosure provides a method of detecting an ADA against an anti-Factor XI and/or anti-Factor XIa antibody or antigen-binding fragment thereof, that bind human, rabbit, baboon, and cynomolgus monkey FXI and/or FXIa. In certain embodiments, the anti-Factor FXI and/or anti-Factor FXIa antibodies may comprise a heavy chain variable domain (VH) having an amino acid sequence of SEQ ID NOs: 9 or 29. In certain embodiments, the antibodies comprise a VH having an amino acid sequence of SEQ ID NO:29. The present disclosure also provides a method of detecting an ADA against anti-Factor XI and/or anti-Factor XIa antibodies or antigen-binding fragments thereof, wherein the antibodies specifically bind to a FXI and/or FXIa protein, and comprise a VH CDR having an amino acid sequence of any one of the VH CDRs listed in Table 1, infra. In particular, the present disclosure provides a method of detecting an ADA against anti-Factor XI and/or anti-Factor XIa antibodies or antigen-binding fragments thereof, wherein the antibodies specifically bind to an FXI and/or FXIa protein (e.g., human, rabbit, baboon, and cynomolgus monkey FXI and/or FXIa), and comprise (or alternatively, consist of) one, two, three, or more VH CDRs having an amino acid sequence of any of the VH CDRs listed in Table 1, infra. (see PCT International Patent Application No. PCT/M2016/053790 filed on Jun. 24, 2016, and published as WO2016/207858, which is hereby incorporated by reference in its entirety).

In some embodiments, the present disclosure provides a method of detecting an ADA against anti-Factor XI and/or anti-Factor XIa antibodies or antigen-binding fragments thereof, wherein the antibodies specifically bind to a FXI and/or FXIa protein (e.g., human, rabbit, baboon, and cynomolgus monkey FXI and/or FXIa), and comprise a light chain variable domain (VL) having an amino acid sequence of SEQ ID NOs: 19 or 39. In certain embodiments, the antibodies comprise a VL having an amino acid sequence of SEQ ID NO:39. The present disclosure also provides a method of detecting an ADA against anti-Factor XI and/or anti-Factor XIa antibodies or antigen-binding fragments thereof, wherein the antibodies specifically bind to an FXI and/or FXIa protein (e.g., human, rabbit, baboon, and cynomolgus monkey FXI and/or FXIa), and comprise a VL CDR having an amino acid sequence of any one of the VL CDRs listed in Table 1, infra. In particular, the present disclosure provides a method of detecting an ADA against anti-Factor XI and/or anti-Factor XIa antibodies or antigen-binding fragments thereof, wherein the antibodies specifically bind to an FXIa protein (e.g., human, rabbit, baboon, and cynomolgus monkey FXI and/or FXIa), and comprise (or alternatively, consist of) one, two, three or more VL CDRs having an amino acid sequence of any of the VL CDRs listed in Table 1, infra.

In some embodiments, other anti-Factor XI and/or anti-Factor XIa antibodies are used in a method of detecting an ADA described herein (e.g., method of detecting an ADA against anti-Factor XI and/or anti-Factor XIa antibodies or antigen-binding fragments thereof), and may include amino acids that have been mutated, yet have at least 60, 70, 80, 85, 90 or 95 percent identity in the CDR regions with the CDR regions depicted in the sequences described in Table 1. In some embodiments, the antibodies include mutant amino acid sequences wherein no more than 1, 2, 3, 4 or 5 amino acids have been mutated in the CDR regions when compared with the CDR regions depicted in the sequence described in Table 1.

TABLE 1
Examples of FXI/FXIa Antibodies, Fabs and FXI/FXIa Proteins

| | Sequence | |
|---|---|---|
| | Identifier | |
| | (SEQ ID | |
| Sequence Description | NO:) | Amino acid or polynucleotide sequence |
| Human FXIa full- | 1 | MIFLYQVVHFILFTSVSGECVTQLLKDTCFEGGDIT |
| length protein | | TVFTPSAKYCQVVCTYHPRCLLFTFTAESPSEDPTR |
| sequence (NCBI | | WFTCVLKDSVTETLPRVNRTAAISGYSFKQCSHQI |
| Reference Sequence: | | SACNKDIYVDLDMKGINYNSSVAKSAQECQERCT |
| AAA51985) | | DDVHCHFFTYATRQFPSLEHRNICLLKHTQTGTPT |
| | | RITKLDKVVSGFSLKSCALSNLACIRDIFPNTVFAD |
| | | SNIDSVMAPDAFVSGRICTHHPGCLFFTFFSQEWP |
| | | KESQRNLCLLKTSESGLPSTRIKKSKALSGFSLQSC |
| | | RHSIPVFCHSSFYHDTDFLGEELDIVAAKSHEACQ |
| | | KLCTNAVRCQFFTYTPAQASCNEGKGKCYLKLSS |
| | | NGSPTKILHGRGGISGYTLRLCKMDNECTTKIKPRI |
| | | VGGTASVRGEWPWQVTLHTTSPTQRHLCGGSIIG |
| | | NQWILTAAHCFYGVESPKILRVYSGILNQSEIKEDT |
| | | SFFGVQEIIIHDQYKMAESGYDIALLKLETTVNYTD |
| | | SQRPICLPSKGDRNVIYTDCWVTGWGYRKLRDKI |
| | | QNTLQKAKIPLVTNEECQKRYRGHKITHKMICAG |
| | | YREGGKDACKGDSGGPLSCKHNEVWHLVGITSW |
| | | GEGCAQRERPGVYTNVVEYVDWILEKTQAV |
| Human FXIa full- | 2 | AGGCACACAGGCAAAATCAAGTTCTACATCTGT |
| length nucleotide | | CCCTGTGTATGTCACTTGTTTGAATACGAAATAA |
| sequence (NCBI | | AATTAAAAAAATAAATTCAGTGTATTGAGAAAG |
| Reference Sequence: | | CAAGCAATTCTCTCAAGGTATATTTCTGACATAC |
| NM_000128.3) | | TAAGATTTTAACGACTTTCACAAATATGCTGTAC |
| | | TGAGAGAGAATGTTACATAACATTGAGAACTAG |
| | | TACAAGTAAATATTAAAGTGAAGTGACCATTTC |
| | | CTACACAAGCTCATTCAGAGGAGGATGAAGACC |
| | | ATTTTGGAGGAAGAAAAGCACCCTTATTAAGAA |
| | | TTGCAGCAAGTAAGCCAACAAGGTCTTTTCAGG |
| | | ATGATTTTCTTATATCAAGTGGTACATTTCATTT |
| | | TATTTACTTCAGTTTCTGGTGAATGTGTGACTCA |
| | | GTTGTTGAAGGACACCTGCTTTGAAGGAGGGGA |
| | | CATTACTACGGTCTTCACACCAAGCGCCAAGTA |
| | | CTGCCAGGTAGTCTGCACTTACCACCCAAGATG |
| | | TTTACTCTTCACTTTCACGGCGGAATCACCATCT |
| | | GAGGATCCCACCCGATGGTTTACTTGTGTCCTGA |
| | | AAGACAGTGTTACAGAAACACTGCCAAGAGTGA |
| | | ATAGGACAGCAGCGATTTCTGGGTATTCTTTCAA |
| | | GCAATGCTCACACCAAATAAGCGCTTGCAACAA |
| | | AGACATTTATGTGGACCTAGACATGAAGGGCAT |
| | | AAACTATAACAGCTCAGTTGCCAAGAGTGCTCA |
| | | AGAATGCCAAGAAAGATGCACGGATGACGTCCA |
| | | CTGCCACTTTTTCACGTACGCCACAAGGCAGTTT |
| | | CCCAGCCTGGAGCATCGTAACATTTGTCTACTGA |
| | | AGCACACCCAAACAGGGACACCAACCAGAATA |
| | | ACGAAGCTCGATAAAGTGGTGTCTGGATTTTCA |
| | | CTGAAATCCTGTGCACTTTCTAATCTGGCTTGTA |
| | | TTAGGGACATTTTCCCTAATACGGTGTTTGCAGA |
| | | CAGCAACATCGACAGTGTCATGGCTCCCGATGC |
| | | TTTTGTCTGTGGCCGAATCTGCACTCATCATCCC |
| | | GGTTGCTTGTTTTTTACCTTCTTTTCCCAGGAATG |
| | | GCCCAAAGAATCTCAAAGAAATCTTTGTCTCCTT |
| | | AAAACATCTGAGAGTGGATTGCCCAGTACACGC |
| | | ATTAAAAAGAGCAAAGCTCTTTCTGGTTTCAGTC |
| | | TACAAAGCTGCAGGCACAGCATCCCAGTGTTCT |
| | | GCCATTCTTCATTTTACCATGACACTGATTTCTT |
| | | GGGAGAAGAACTGGATATTGTTGCTGCAAAAAG |
| | | TCACGAGGCCTGCCAGAAACTGTGCACCAATGC |
| | | CGTCCGCTGCCAGTTTTTTACCTATACCCCAGCC |
| | | CAAGCATCCTGCAACGAAGGGAAGGGCAAGTGT |
| | | TACTTAAAGCTTTCTTCAAACGGATCTCCAACTA |
| | | AAATACTTCACGGGAGAGGAGGCATCTCTGGAT |
| | | ACACATTAAGGTTGTGTAAAATGGATAATGAGT |
| | | GTACCACCAAAATCAAGCCCAGGATCGTTGGAG |
| | | GAACTGCGTCTGTTCGTGGTGAGTGGCCGTGGC |
| | | AGGTGACCCTGCACACAACCTCACCCACTCAGA |
| | | GACACCTGTGTGGAGGCTCCATCATTGGAAACC |
| | | AGTGGATATTAACAGCCGCTCACTGTTTCTATGG |
| | | GGTAGAGTCACCTAAGATTTTGCGTGTCTACAGT |
| | | GGCATTTTAAATCAATCTGAAATAAAAGAGGAC |
| | | ACATCTTTCTTTGGGGTTCAAGAAATAATAATCC |
| | | ATGATCAGTATAAAATGGCAGAAAGCGGGTATG |
| | | ATATTGCCTTGTTGAAACTGGAAACCACAGTGA |
| | | ATTACACAGATTCTCAACGACCCATATGCCTGCC |
| | | TTCCAAAGGAGATAGAAATGTAATATACACTGA |
| | | TTGCTGGGTGACTGGATGGGGGTACAGAAAACT |
| | | AAGAGACAAAATACAAAATACTCTCCAGAAAGC |
| | | CAAGATACCCTTAGTGACCAACGAAGAGTGCCA |
| | | GAAGAGATACAGAGGACATAAAATAACCCATA |
| | | AGATGATCTGTGCCGGCTACAGGGAAGGAGGGA |
| | | AGGACGCTTGCAAGGGAGATTCGGGAGGCCCTC |
| | | TGTCCTGCAAACACAATGAGGTCTGGCATCTGG |
| | | TAGGCATCACGAGCTGGGGCGAAGGCTGTGCTC |
| | | AAAGGGAGCGGCCAGGTGTTTACACCAACGTGG |
| | | TCGAGTACGTGGACTGGATTCTGGAGAAAACTC |
| | | AAGCAGTGTGAATGGGTTCCCAGGGGCCATTGG |
| | | AGTCCCTGAAGGACCCAGGATTTGCTGGGAGAG |
| | | GGTGTTGAGTTCACTGTGCCAGCATGCTTCCTCC |
| | | ACAGTAACACGCTGAAGGGGCTTGGTGTTTGTA |
| | | AGAAAATGCTAGAAGAAAACAAACTGTCACAA |
| | | GTTGTTATGTCCAAAACTCCCGTTCTATGATCGT |
| | | TGTAGTTTGTTTGAGCATTCAGTCTCTTTGTTTTT |
| | | GATCACGCTTCTATGGAGTCCAAGAATTACCAT |
| | | AAGGCAATATTTCTGAAGATTACTATATAGGCA |
| | | GATATAGCAGAAAATAACCAAGTAGTGGCAGTG |
| | | GGGATCAGGCAGAAGAACTGGTAAAAGAAGCC |
| | | ACCATAAATAGATTTGTTCGATGAAAGATGAAA |
| | | ACTGGAAGAAAGGAGAACAAAGACAGTCTTCA |
| | | CCATTTTGCAGGAATCTACACTCTGCCTATGTGA |
| | | ACACATTTCTTTTGTAAAGAAAGAAATTGATTGC |
| | | ATTTAATGGCAGATTTTCAGAATAGTCAGGAAT |
| | | TCTTGTCATTTCCATTTTAAAATATATATTAAAA |
| | | AAAATCAGTTCGAGTAGACACGAGCTAAGAGTG |
| | | AATGTGAAGATAACAGAATTTCTGTGTGGAAGA |
| | | GGATTACAAGCAGCAATTTACCTGGAAGTGATA |
| | | CCTTAGGGGCAATCTTGAAGATACACTTTCCTGA |
| | | AAAATGATTTGTGATGGATTGTATATTTATTTAA |
| | | AATATCTTGGGAGGGGAGGCTGATGGAGATAGG |
| | | GAGCATGCTCAAACCTCCCTAAGACAAGCTGCT |
| | | GCTGTGACTATGGGCTCCCAAAGAGCTAGATCG |
| | | TATATTTATTTGACAAAAATCACCATAGACTGCA |
| | | TCCATACTACAGAGAAAAAACAATTAGGGCGCA |
| | | AATGGATAGTTACAGTAAAGTCTTCAGCAAGCA |
| | | GCTGCCTGTATTCTAAGCACTGGGATTTTCTGTT |
| | | TCGTGCAAATATTTATCTCATTATTGTTGTGATC |
| | | TAGTTCAATAACCTAGAATTTGAATTGTCACCAC |
| | | ATAGCTTTCAATCTGTGCCAACAACTATACAATT |
| | | CATCAAGTGTG |

Antibody 2

| | | |
|---|---|---|
| HCDR1 (Kabat) | 3 | TAAMS |
| HCDR2 (Kabat) | 4 | GISGSGSSTYYADSVKG |
| HCDR3 (Kabat) | 5 | ELSYLYSGYYFDY |
| HCDR1 (Chothia) | 6 | GFTFSTA |
| HCDR2 (Chothia) | 7 | SGSGSS |
| HCDR3 (Chothia) | 8 | ELSYLYSGYYFDY |
| HCDR1 (IMGT) | 43 | GFTFSTAA |
| HCDR2 (IMGT) | 44 | ISGSGSST |
| HCDR3 (IMGT) | 45 | ARELSYLYSGYYFDY |
| HCDR1 (Combined) | 46 | GFTFSTAAMS |
| HCDR2 (Combined) | 4 | GISGSGSSTYYADSVKG |
| HCDR3 (Combined) | 5 | ELSYLYSGYYFDY |
| VH | 9 | QVQLLESGGGLVQPGGSLRLSCAASGFTFSTAAMS |
| | | WVRQAPGKGLEWVSGISGSGSSTYYADSVKGRFT |
| | | ISRDNSKNTLYLQMNSLRAEDTAVYYCARELSYL |
| | | YSGYYFDYWGQGTLVTVSS |
| DNA encoding VH | 10 | CAGGTGCAATTGCTGGAAAGCGGCGGTGGCCTG |
| | | GTGCAGCCGGGTGGCAGCCTGCGTCTGAGCTGC |
| | | GCGGCGTCCGGATTCACCTTTTCTACTGCTGCTA |
| | | TGTCTTGGGTGCGCCAGGCCCCGGGCAAAGGTC |
| | | TCGAGTGGGTTTCCGGTATCTCTGGTTCTGGTTC |
| | | TTCTACCTACTATGCGGATAGCGTGAAAGGCCG |
| | | CTTTACCATCAGCCGCGATAATTCGAAAAACAC |
| | | CCTGTATCTGCAAATGAACAGCCTGCGTGCGGA |
| | | AGATACGGCCGTGTATTATTGCGCGCGTGAACT |
| | | GTCTTACCTGTACTCTGGTTACTACTTCGATTAC |
| | | TGGGGCCAAGGCACCCTGGTGACTGTTAGCTCA |
| Heavy Chain | 11 | QVQLLESGGGLVQPGGSLRLSCAASGFTFSTAAMS |
| | | WVRQAPGKGLEWVSGISGSGSSTYYADSVKGRFT |
| | | ISRDNSKNTLYLQMNSLRAEDTAVYYCARELSYL |
| | | YSGYYFDYWGQGTLVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICNVN |
| | | HKPSNTKVDKRVEPKSCDKTHTCPPCPAPEAAGG |
| | | PSVFLFPPKPKDTLMISRTPEVTCVVVDVSHEDPEV |
| | | KFNWYVDGVEVHNAKTKPREEQYNSTYRVVSVL |
| | | TVLHQDWLNGKEYKCKVSNKALPAPIEKTISKAK |
| | | GQPREPQVYTLPPSREEMTKNQVSLTCLVKGFYPS |
| | | DIAVEWESNGQPENNYKTTPPVLDSDGSFFLYSKL |
| | | TVDKSRWQQGNVFSCSVMHEALHNHYTQKSLSLS |
| | | PGK |
| DNA encoding Heavy | 12 | CAGGTGCAATTGCTGGAAAGCGGCGGTGGCCTG |
| Chain | | GTGCAGCCGGGTGGCAGCCTGCGTCTGAGCTGC |
| | | GCGGCGTCCGGATTCACCTTTTCTACTGCTGCTA |
| | | TGTCTTGGGTGCGCCAGGCCCCGGGCAAAGGTC |
| | | TCGAGTGGGTTTCCGGTATCTCTGGTTCTGGTTC |
| | | TTCTACCTACTATGCGGATAGCGTGAAAGGCCG |
| | | CTTTACCATCAGCCGCGATAATTCGAAAAACAC |
| | | CCTGTATCTGCAAATGAACAGCCTGCGTGCGGA |
| | | AGATACGGCCGTGTATTATTGCGCGCGTGAACT |
| | | GTCTTACCTGTACTCTGGTTACTACTTCGATTAC |
| | | TGGGGCCAAGGCACCCTGGTGACTGTTAGCTCA |
| | | GCCTCCACCAAGGGTCCATCGGTCTTCCCCCTGG |
| | | CACCCTCCTCCAAGAGCACCTCTGGGGGCACAG |
| | | CGGCCCTGGGCTGCCTGGTCAAGGACTACTTCC |
| | | CCGAACCGGTGACGGTGTCGTGGAACTCAGGCG |
| | | CCCTGACCAGCGGCGTGCACACCTTCCCGGCTG |
| | | TCCTACAGTCCTCAGGACTCTACTCCCTCAGCAG |
| | | CGTGGTGACCGTGCCCTCCAGCAGCTTGGGCAC |
| | | CCAGACCTACATCTGCAACGTGAATCACAAGCC |
| | | CAGCAACACCAAGGTGGACAAGAGAGTTGAGC |
| | | CCAAATCTTGTGACAAAACTCACACATGCCCAC |
| | | CGTGCCCAGCACCTGAAGCAGCGGGGGGACCGT |
| | | CAGTCTTCCTCTTCCCCCCAAAACCCAAGGACAC |
| | | CCTCATGATCTCCCGGACCCCTGAGGTCACATGC |
| | | GTGGTGGTGGACGTGAGCCACGAAGACCCTGAG |
| | | GTCAAGTTCAACTGGTACGTGGACGGCGTGGAG |
| | | GTGCATAATGCCAAGACAAAGCCGCGGGAGGA |
| | | GCAGTACAACAGCACGTACCGGGTGGTCAGCGT |
| | | CCTCACCGTCCTGCACCAGGACTGGCTGAATGG |
| | | CAAGGAGTACAAGTGCAAGGTCTCCAACAAAGC |
| | | CCTCCCAGCCCCCATCGAGAAAACCATCTCCAA |
| | | AGCCAAAGGGCAGCCCCGAGAACCACAGGTGT |
| | | ACACCCTGCCCCCATCCCGGGAGGAGATGACCA |
| | | AGAACCAGGTCAGCCTGACCTGCCTGGTCAAAG |
| | | GCTTCTATCCCAGCGACATCGCCGTGGAGTGGG |
| | | AGAGCAATGGGCAGCCGGAGAACAACTACAAG |
| | | ACCACGCCTCCCGTGCTGGACTCCGACGGCTCCT |
| | | TCTTCCTCTACAGCAAGCTCACCGTGGACAAGA |
| | | GCAGGTGGCAGCAGGGGAACGTCTTCTCATGCT |
| | | CCGTGATGCATGAGGCTCTGCACAACCACTACA |
| | | CGCAGAAGAGCCTCTCCCTGTCTCCGGGTAAA |
| LCDR1 (Kabat) | 13 | SGSSSNIGSNDVS |
| LCDR2 (Kabat) | 14 | KNYNRPS |
| LCDR3 (Kabat) | 15 | SAWDQRQFDVV |
| LCDR1 (Chothia) | 16 | SSSNIGSND |
| LCDR2 (Chothia) | 17 | KNY |
| LCDR3 (Chothia) | 18 | WDQRQFDV |
| LCDR1 (IMGT) | 47 | SSNIGSND |
| LCDR2 (IMGT) | 37 | KNY |
| LCDR3 (IMGT) | 15 | SAWDQRQFDVV |
| LCDR1 (Combined) | 33 | SGSSSNIGSNDVS |
| LCDR2 (Combined) | 14 | KNYNRPS |
| LCDR3 (Combined) | 15 | SAWDQRQFDVV |
| VL | 19 | DIVLTQPPSVSGAPGQRVTISCSGSSSNIGSNDVSW |
| | | YQQLPGTAPKLLIYKNYNRPSGVPDRFSGSKSGTS |
| | | ASLAITGLQAEDEADYYCSAWDQRQFDVVFGGGT |
| | | KLTVL |
| DNA encoding VL | 20 | GATATCGTGCTGACCCAGCCGCCGAGCGTGAGC |
| | | GGTGCACCGGGCCAGCGCGTGACCATTAGCTGT |
| | | AGCGGCAGCAGCAGCAACATTGGTTCTAACGAC |
| | | GTGTCTTGGTACCAGCAGCTGCCGGGCACGGCG |
| | | CCGAAACTGCTGATCTACAAAAACTACAACCGC |
| | | CCGAGCGGCGTGCCGGATCGCTTTAGCGGATCC |
| | | AAAAGCGGCACCAGCGCCAGCCTGGCGATTACC |
| | | GGCCTGCAAGCAGAAGACGAAGCGGATTATTAC |
| | | TGCTCTGCTTGGGACCAGCGTCAGTTCGACGTTG |
| | | TGTTTGGCGGCGGCACGAAGTTAACCGTCCTA |
| Light Chain | 21 | DIVLTQPPSVSGAPGQRVTISCSGSSSNIGSNDVSW |
| | | YQQLPGTAPKLLIYKNYNRPSGVPDRFSGSKSGTS |
| | | ASLAITGLQAEDEADYYCSAWDQRQFDVVFGGGT |
| | | KLTVLGQPKAAPSVTLFPPSSEELQANKATLVCLIS |
| | | DFYPGAVTVAWKADSSPVKAGVETTTPSKQSNNK |
| | | YAASSYLSLTPEQWKSHRSYSCQVTHEGSTVEKT |
| | | VAPTECS |
| DNA encoding Light | 22 | GATATCGTGCTGACCCAGCCGCCGAGCGTGAGC |
| Chain | | GGTGCACCGGGCCAGCGCGTGACCATTAGCTGT |
| | | AGCGGCAGCAGCAGCAACATTGGTTCTAACGAC |
| | | GTGTCTTGGTACCAGCAGCTGCCGGGCACGGCG |
| | | CCGAAACTGCTGATCTACAAAAACTACAACCGC |
| | | CCGAGCGGCGTGCCGGATCGCTTTAGCGGATCC |
| | | AAAAGCGGCACCAGCGCCAGCCTGGCGATTACC |
| | | GGCCTGCAAGCAGAAGACGAAGCGGATTATTAC |
| | | TGCTCTGCTTGGGACCAGCGTCAGTTCGACGTTG |
| | | TGTTTGGCGGCGGCACGAAGTTAACCGTCCTAG |
| | | GTCAGCCCAAGGCTGCCCCCTCGGTCACTCTGTT |
| | | CCCGCCCTCCTCTGAGGAGCTTCAAGCCAACAA |
| | | GGCCACACTGGTGTGTCTCATAAGTGACTTCTAC |
| | | CCGGGAGCCGTGACAGTGGCCTGGAAGGCAGAT |
| | | AGCAGCCCCGTCAAGGCGGGAGTGGAGACCACC |
| | | ACACCCTCCAAACAAAGCAACAACAAGTACGCG |
| | | GCCAGCAGCTATCTGAGCCTGACGCCTGAGCAG |
| | | TGGAAGTCCCACAGAAGCTACAGCTGCCAGGTC |
| | | ACGCATGAAGGGAGCACCGTGGAGAAGACAGT |
| | | GGCCCCTACAGAATGTTCA |

Antibody 1

| | | |
|---|---|---|
| HCDR1 (Kabat) | 23 | TAAMS |
| HCDR2 (Kabat) | 24 | GISGSGSSTYYADSVKG |
| HCDR3 (Kabat) | 25 | ELSYLYSGYYFDY |
| HCDR1 (Chothia) | 26 | GFTFSTA |
| HCDR2 (Chothia) | 27 | SGSGSS |
| HCDR3 (Chothia) | 28 | ELSYLYSGYYFDY |
| HCDR1 (IMGT) | 43 | GFTFSTAA |
| HCDR2 (IMGT) | 44 | ISGSGSST |
| HCDR3 (IMGT) | 45 | ARELSYLYSGYYFDY |
| HCDR1 (Combined) | 46 | GFTFSTAAMS |
| HCDR2 (Combined) | 4 | GISGSGSSTYYADSVKG |
| HCDR3 (Combined) | 5 | ELSYLYSGYYFDY |
| VH | 29 | QVQLLESGGGLVQPGGSLRLSCAASGFTFSTAAMS |
| | | WVRQAPGKGLEWVSGISGSGSSTYYADSVKGRFT |
| | | ISRDNSKNTLYLQMNSLRAEDTAVYYCARELSYL |
| | | YSGYYFDYWGQGTLVTVSS |
| DNA encoding VH | 30 | CAGGTGCAGCTGCTGGAATCAGGCGGCGGACTG |
| | | GTGCAGCCTGGCGGTAGCCTGAGACTGAGCTGC |
| | | GCTGCTAGTGGCTTCACCTTTAGCACCGCCGCTA |
| | | TGAGCTGGGTTCGACAGGCCCCAGGGAAAGGCC |
| | | TCGAGTGGGTCTCAGGGATTAGCGGTAGCGGCT |
| | | CTAGCACCTACTACGCCGATAGCGTGAAGGGCC |
| | | GGTTCACTATCTCTAGGGATAACTCTAAGAACA |
| | | CCCTGTACCTGCAGATGAATAGCCTGAGAGCCG |
| | | AGGACACCGCCGTCTACTACTGCGCTAGAGAGC |
| | | TGAGCTACCTGTATAGCGGCTACTACTTCGACTA |
| | | CTGGGGTCAAGGCACCCTGGTCACCGTGTCTAG |
| | | C |
| Heavy Chain | 31 | QVQLLESGGGLVQPGGSLRLSCAASGFTFSTAAMS |
| | | WVRQAPGKGLEWVSGISGSGSSTYYADSVKGRFT |
| | | ISRDNSKNTLYLQMNSLRAEDTAVYYCARELSYL |
| | | YSGYYFDYWGQGTLVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICNVN |
| | | HKPSNTKVDKRVEPKSCDKTHTCPPCPAPELLGGP |
| | | SVFLFPPKPKDTLMISRTPEVTCVVVAVSHEDPEV |
| | | KFNWYVDGVEVHNAKTKPREEQYNSTYRVVSVL |
| | | TVLHQDWLNGKEYKCKVSNKALAAPIEKTISKAK |
| | | GQPREPQVYTLPPSREEMTKNQVSLTCLVKGFYPS |
| | | DIAVEWESNGQPENNYKTTPPVLDSDGSFFLYSKL |
| | | TVDKSRWQQGNVFSCSVMHEALHNHYTQKSLSLS |
| | | PGK |
| DNA encoding Heavy | 32 | CAGGTGCAGCTGCTGGAATCAGGCGGCGGACTG |
| Chain | | GTGCAGCCTGGCGGTAGCCTGAGACTGAGCTGC |
| | | GCTGCTAGTGGCTTCACCTTTAGCACCGCCGCTA |
| | | TGAGCTGGGTTCGACAGGCCCCAGGGAAAGGCC |
| | | TCGAGTGGGTCTCAGGGATTAGCGGTAGCGGCT |
| | | CTAGCACCTACTACGCCGATAGCGTGAAGGGCC |
| | | GGTTCACTATCTCTAGGGATAACTCTAAGAACA |
| | | CCCTGTACCTGCAGATGAATAGCCTGAGAGCCG |
| | | AGGACACCGCCGTCTACTACTGCGCTAGAGAGC |
| | | TGAGCTACCTGTATAGCGGCTACTACTTCGACTA |
| | | CTGGGGTCAAGGCACCCTGGTCACCGTGTCTAG |
| | | CGCTAGCACTAAGGGCCCCTCCGTGTTCCCTCTG |
| | | GCCCCTTCCAGCAAGTCTACCTCCGGCGGCACA |
| | | GCTGCTCTGGGCTGCCTGGTCAAGGACTACTTCC |
| | | CTGAGCCTGTGACAGTGTCCTGGAACTCTGGCG |
| | | CCCTGACCTCTGGCGTGCACACCTTCCCTGCCGT |
| | | GCTGCAGTCCTCCGGCCTGTACTCCCTGTCCTCC |
| | | GTGGTCACAGTGCCTTCAAGCAGCCTGGGCACC |
| | | CAGACCTATATCTGCAACGTGAACCACAAGCCT |
| | | TCCAACACCAAGGTGGACAAGCGGGTGGAGCCT |
| | | AAGTCCTGCGACAAGACCCACACCTGTCCTCCC |
| | | TGCCCTGCTCCTGAACTGCTGGGCGGCCCTTCTG |
| | | TGTTCCTGTTCCCTCCAAAGCCCAAGGACACCCT |
| | | GATGATCTCCCGGACCCCTGAAGTGACCTGCGT |
| | | GGTGGTGGCCGTGTCCCACGAGGATCCTGAAGT |
| | | GAAGTTCAATTGGTACGTGGACGGCGTGGAGGT |
| | | GCACAACGCCAAGACCAAGCCTCGGGAGGAAC |
| | | AGTACAACTCCACCTACCGGGTGGTGTCCGTGC |
| | | TGACCGTGCTGCACCAGGACTGGCTGAACGGCA |
| | | AAGAGTACAAGTGCAAAGTCTCCAACAAGGCCC |
| | | TGGCCGCCCCTATCGAAAAGACAATCTCCAAGG |
| | | CCAAGGGCCAGCCTAGGGAACCCCAGGTGTACA |
| | | CCCTGCCACCCAGCCGGGAGGAAATGACCAAGA |
| | | ACCAGGTGTCCCTGACCTGTCTGGTCAAGGGCTT |
| | | CTACCCTTCCGATATCGCCGTGGAGTGGGAGTCT |
| | | AACGGCCAGCCTGAGAACAACTACAAGACCACC |
| | | CCTCCTGTGCTGGACTCCGACGGCTCCTTCTTCC |
| | | TGTACTCCAAACTGACCGTGGACAAGTCCCGGT |
| | | GGCAGCAGGGCAACGTGTTCTCCTGCTCCGTGA |
| | | TGCACGAGGCCCTGCACAACCACTACACCCAGA |
| | | AGTCCCTGTCCCTGTCTCCCGGCAAG |
| LCDR1 (Kabat) | 33 | SGSSSNIGSNDVS |
| LCDR2 (Kabat) | 34 | KNYNRPS |
| LCDR3 (Kabat) | 35 | SAWDQRQFDVV |
| LCDR1 (Chothia) | 36 | SSSNIGSND |
| LCDR2 (Chothia) | 37 | KNY |
| LCDR3 (Chothia) | 38 | WDQRQFDV |
| LCDR1 (IMGT) | 47 | SSNIGSND |
| LCDR2 (IMGT) | 37 | KNY |
| LCDR3 (IMGT) | 15 | SAWDQRQFDVV |
| LCDR1 (Combined) | 33 | SGSSSNIGSNDVS |
| LCDR2 (Combined) | 14 | KNYNRPS |
| LCDR3 (Combined) | 15 | SAWDQRQFDVV |
| VL | 39 | QSVLTQPPSASGTPGQRVTISCSGSSSNIGSNDVSW |
| | | YQQLPGTAPKLLIYKNYNRPSGVPDRFSGSKSGTS |
| | | ASLAISGLQSEDEADYYCSAWDQRQFDVVFGGGT |
| | | KLTVL |
| DNA encoding VL | 40 | CAGTCAGTCCTGACTCAGCCCCCTAGCGCTAGT |
| | | GGCACCCCTGGTCAAAGAGTGACTATTAGCTGT |
| | | AGCGGCTCTAGCTCTAATATCGGCTCTAACGAC |
| | | GTCAGCTGGTATCAGCAGCTGCCCGGCACCGCC |
| | | CCTAAGCTGCTGATCTATAAGAACTATAATAGG |
| | | CCTAGCGGCGTGCCCGATAGGTTTAGCGGATCT |
| | | AAATCAGGGACTTCTGCTAGTCTGGCTATTAGC |
| | | GGCCTGCAGTCAGAGGACGAGGCCGACTACTAC |
| | | TGTAGCGCCTGGGATCAGCGTCAGTTCGACGTG |
| | | GTGTTCGGCGGAGGCACTAAGCTGACCGTGCTG |
| Light Chain | 41 | QSVLTQPPSASGTPGQRVTISCSGSSSNIGSNDVSW |
| | | YQQLPGTAPKLLIYKNYNRPSGVPDRFSGSKSGTS |
| | | ASLAISGLQSEDEADYYCSAWDQRQFDVVFGGGT |
| | | KLTVLGQPKAAPSVTLFPPSSEELQANKATLVCLIS |
| | | DFYPGAVTVAWKADSSPVKAGVETTTPSKQSNNK |
| | | YAASSYLSLTPEQWKSHRSYSCQVTHEGSTVEKT |
| | | VAPTECS |
| DNA encoding Light | 42 | CAGTCAGTCCTGACTCAGCCCCCTAGCGCTAGT |
| Chain | | GGCACCCCTGGTCAAAGAGTGACTATTAGCTGT |
| | | AGCGGCTCTAGCTCTAATATCGGCTCTAACGAC |
| | | GTCAGCTGGTATCAGCAGCTGCCCGGCACCGCC |
| | | CCTAAGCTGCTGATCTATAAGAACTATAATAGG |
| | | CCTAGCGGCGTGCCCGATAGGTTTAGCGGATCT |
| | | AAATCAGGGACTTCTGCTAGTCTGGCTATTAGC |
| | | GGCCTGCAGTCAGAGGACGAGGCCGACTACTAC |
| | | TGTAGCGCCTGGGATCAGCGTCAGTTCGACGTG |
| | | GTGTTCGGCGGAGGCACTAAGCTGACCGTGCTG |
| | | GGTCAACCTAAGGCTGCCCCCAGCGTGACCCTG |
| | | TTCCCCCCCAGCAGCGAGGAGCTGCAGGCCAAC |
| | | AAGGCCACCCTGGTGTGCCTGATCAGCGACTTC |
| | | TACCCAGGCGCCGTGACCGTGGCCTGGAAGGCC |
| | | GACAGCAGCCCCGTGAAGGCCGGCGTGGAGACC |
| | | ACCACCCCCAGCAAGCAGAGCAACAACAAGTAC |
| | | GCCGCCAGCAGCTACCTGAGCCTGACCCCCGAG |
| | | CAGTGGAAGAGCCACAGGTCCTACAGCTGCCAG |
| | | GTGACCCACGAGGGCAGCACCGTGGAAAAGAC |
| | | CGTGGCCCCAACCGAGTGCAGC |

In some embodiments, anti-Factor XI and/or anti-Factor XIa antibodies that are used in a method of detecting an ADA described herein (e.g., a method of detecting an ADA against anti-Factor XI and/or anti-Factor XIa antibodies or antigen-binding fragments thereof) comprise an amino acid sequence in which the amino acids have been mutated, yet have at least 60, 65, 70, 75, 80, 85, 90, or 95 percent identity to the sequences described in Table 1. In some embodiments, the anti-Factor XI and/or anti-Factor XIa antibodies include mutant amino acid sequences wherein no more than 1, 2, 3, 4 or 5 amino acids have been mutated in the variable regions when compared with the variable regions depicted in the sequence described in Table 1, while retaining substantially the same antigen binding activity.

Since each of these antibodies can bind to FXI and/or FXIa, the VH, VL, full length light chain, and full length heavy chain sequences (amino acid sequences and the nucleotide sequences encoding the amino acid sequences) can be “mixed and matched” to create other FXI and/or FXIa-binding antibodies of the present disclosure. Such “mixed and matched” FXI and/or FXIa-binding antibodies can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full length heavy chain sequence from a particular full length heavy chain/full length light chain pairing should be replaced with a structurally similar full length heavy chain sequence. Likewise, a VL sequence from a particular VH/VL pairing should be replaced with a structurally similar VL sequence. Likewise a full length light chain sequence from a particular full length heavy chain/full length light chain pairing should be replaced with a structurally similar full length light chain sequence.

Accordingly, for use in the methods described herein (e.g., a method of detecting an ADA against anti-Factor XI and/or anti-Factor XIa antibodies or antigen-binding fragments thereof), the present disclosure provides an isolated antibody or antigen binding region thereof having: a heavy chain variable domain comprising an amino acid sequence selected from the group consisting of SEQ ID NOs: 9 and 29, and a light chain variable domain comprising an amino acid sequence selected from the group consisting of SEQ ID NOs: 19 and 39, wherein the antibody specifically binds to FXI and/or FXIa (e.g., human, rabbit, baboon, and cynomolgus monkey FXIa).

In certain embodiments, the present disclosure provides an isolated antibody or antigen binding fragment thereof having a heavy chain variable domain and a light chain variable domain comprising amino acid sequences selected from SEQ ID NOs: 9 and 29; or 19 and 39, respectively, for use in a method of detecting an ADA against anti-Factor XI and/or anti-Factor XIa antibodies or antigen-binding fragments thereof.

In certain embodiments for use in the methods described herein (e.g., a method of detecting an ADA against anti-Factor XI and/or anti-Factor XIa antibodies or antigen-binding fragments thereof), an antibody or antigen binding fragment thereof provided herein which specifically binds to human FXI and/or FXIa, comprises a heavy chain variable region comprising the amino acid sequence of SEQ ID NO: 9, and a light chain variable region comprising the amino acid sequence of SEQ ID NO: 19.

In certain embodiments for use in the methods described herein (e.g., a method of detecting an ADA against anti-Factor XI and/or anti-Factor XIa antibodies or antigen-binding fragments thereof), an antibody or antigen binding fragment thereof provided herein which specifically binds to human FXI and/or FXIa, comprises a heavy chain variable region comprising the amino acid sequence of SEQ ID NO: 29, and a light chain variable region comprising the amino acid sequence of SEQ ID NO: 39.

In certain embodiments for use in the methods described herein (e.g., a method of detecting an ADA against anti-Factor XI and/or anti-Factor XIa antibodies or antigen-binding fragments thereof), the present disclosure provides (i) an isolated antibody having: a full length heavy chain comprising an amino acid sequence that has been optimized for expression in a mammalian cell selected from the group consisting of SEQ ID NOs: 11 or 31, and a full length light chain comprising an amino acid sequence that has been optimized for expression in a mammalian cell selected from the group consisting of SEQ ID NOs: 21 or 41; or (ii) a functional protein comprising an antigen binding portion thereof. In certain embodiments, the present disclosure provides an isolated antibody or antigen binding region thereof having a heavy chain and a light chain comprising amino acid sequences selected from SEQ ID NOs: 11 and 31; or 21 and 41, respectively, for use in a method of detecting an ADA against anti-Factor XI and/or anti-Factor XIa antibodies or antigen-binding fragments thereof.

In certain embodiments for use in the methods described herein (e.g., a method of detecting an ADA against anti-Factor XI and/or anti-Factor XIa antibodies or antigen-binding fragments thereof), an antibody or antigen binding fragment thereof provided herein which specifically binds to human FXI and/or FXIa, comprises a heavy chain comprising the amino acid sequence of SEQ ID NO: 11, and a light chain comprising the amino acid sequence of SEQ ID NO: 21.

In certain embodiments for use in the methods described herein (e.g., a method of detecting an ADA against anti-Factor XI and/or anti-Factor XIa antibodies or antigen-binding fragments thereof), an antibody or antigen binding fragment thereof provided herein which specifically binds to human FXI and/or FXIa, comprises a heavy chain variable region comprising the amino acid sequence of SEQ ID NO: 31, and a light chain variable region comprising the amino acid sequence of SEQ ID NO: 41.

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme), Lefranc et al., (2003) Dev. Comp. Immunol., 27, 55-77 (“IMGT” numbering scheme), or the “Combined” system.

For example, under Kabat, the CDR amino acid residues of antibody Antibody 2 in the heavy chain variable domain (VH) are numbered 31-35 (HCDR1), 50-66 (HCDR2), and 99-111 (HCDR3); and the CDR amino acid residues in the light chain variable domain (VL) are numbered 22-35 (LCDR1), 51-57 (LCDR2), and 90-100 (LCDR3). Under Chothia the CDR amino acids in the VH are numbered 26-32 (HCDR1), 52-57 (HCDR2), and 99-111 (HCDR3); and the amino acid residues in VL are numbered 25-33 (LCDR1), 51-53 (LCDR2), and 92-99 (LCDR3). By combining the CDR definitions of both Kabat and Chothia, the CDRs consist of amino acid residues 26-35 (HCDR1), 50-66 (HCDR2), and 99-111 (HCDR3) in human VH and amino acid residues 22-35 (LCDR1), 51-57 (LCDR2), and 90-100 (LCDR3) in human VL. By combining the CDR definitions of both Kabat and Chothia, the “Combined” CDRs consist of amino acid residues 26-35 (HCDR1), 50-66 (HCDR2), and 99-108 (HCDR3) in human VH and amino acid residues 24-38 (LCDR1), 54-60 (LCDR2), and 93-101 (LCDR3) in human VL. As another example, under IMGT, the CDR amino acid residues in the heavy chain variable domain (VH) are numbered 26-33 (HCDR1), 51-58 (HCDR2), and 97-108 (HCDR3); and the CDR amino acid residues in the light chain variable domain (VL) are numbered 27-36 (LCDR1), 54-56 (LCDR2), and 93-101 (LCDR3). Table 1 provides exemplary Kabat, Chothia, Combined, and IMGT HCDR1, HCDR2, HCDR3, LCDR1, LCDR2, and LCDR3 for anti-FXI/FXIa antibodies, e.g., Antibody 2 and Antibody 1. In another aspect, the present disclosure provides FXIa binding antibodies that comprise the heavy chain and light chain CDR1s, CDR2s, and CDR3s as described in Table 1, or combinations thereof. The amino acid sequences of the VH CDR1s of the antibodies are shown in SEQ ID NOs: 3 and 23. The amino acid sequences of the VH CDR2s of the antibodies are shown in SEQ ID NOs: 4 and 24. The amino acid sequences of the VH CDR3s of the antibodies are shown in SEQ ID NOs: 5 and 25. The amino acid sequences of the VL CDR1s of the antibodies are shown in SEQ ID NOs: 13 and 33. The amino acid sequences of the VL CDR2s of the antibodies are shown in SEQ ID NOs: 14 and 34. The amino acid sequences of the VL CDR3s of the antibodies are shown in SEQ ID NOs: 15 and 35. These CDR regions are delineated using the Kabat system.

Alternatively, as defined using the Chothia system (Al-Lazikani et al., (1997) JMB 273, 927-948), the amino acid sequences of the VH CDR1s of the antibodies are shown in SEQ ID NOs: 6 and 26. The amino acid sequences of the VH CDR2s of the antibodies and are shown in SEQ ID NOs: 7 and 27. The amino acid sequences of the VH CDR3s of the antibodies are shown in SEQ ID NOs: 8 and 28. The amino acid sequences of the VL CDR1s of the antibodies are shown in SEQ ID NOs: 16 and 36. The amino acid sequences of the VL CDR2s of the antibodies are shown in SEQ ID NOs: 17 and 37. The amino acid sequences of the VL CDR3s of the antibodies are shown in SEQ ID NOs: 18 and 38.

Alternatively, as defined using the Combined system, the amino acid sequences of the VH CDR1 of the antibodies are shown in SEQ ID NO: 46. The amino acid sequences of the VH CDR2 of the antibodies and are shown in SEQ ID NO: 4. The amino acid sequences of the VH CDR3 of the antibodies are shown in SEQ ID NO: 5. The amino acid sequences of the VL CDR1 of the antibodies are shown in SEQ ID NO: 33. The amino acid sequences of the VL CDR2 of the antibodies are shown in SEQ ID NO: 14. The amino acid sequences of the VL CDR3 of the antibodies are shown in SEQ ID NO: 15.

Alternatively, as defined using the IMGT numbering scheme, the amino acid sequences of the VH CDR1 of the antibodies are shown in SEQ ID NO: 43. The amino acid sequences of the VH CDR2 of the antibodies and are shown in SEQ ID NO: 44. The amino acid sequences of the VH CDR3 of the antibodies are shown in SEQ ID NO: 45. The amino acid sequences of the VL CDR1 of the antibodies are shown in SEQ ID NO: 47. The amino acid sequences of the VL CDR2 of the antibodies are shown in SEQ ID NO: 37. The amino acid sequences of the VL CDR3 of the antibodies are shown in SEQ ID NO: 15.

Given that each of these antibodies can bind to FXI and/or FXIa and that antigen-binding specificity is provided primarily by the CDR1, 2 and 3 regions, the VH CDR1, 2 and 3 sequences and VL CDR1, 2 and 3 sequences can be “mixed and matched” (i.e., CDRs from different antibodies can be mixed and matched, although each antibody preferably contains a VH CDR1, 2 and 3 and a VL CDR1, 2 and 3 to create other FXI and/or FXIa binding molecules of the present disclosure. Such “mixed and matched” FXI and/or FXIa binding antibodies can be tested using the binding assays known in the art and those described in the Examples (e.g., ELISAs, SET, BIACORE™ assays). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein for monoclonal antibodies of the present disclosure. In addition to the foregoing, in one embodiment, the antigen binding fragments of the antibodies described herein can comprise a VH CDR1, 2, and 3, or a VL CDR 1, 2, and 3, wherein the fragment binds to FXI and/or FXIa as a single variable domain. It is noted that the CDR sequences of Antibody 1 and Antibody 2 are identical.

In certain embodiments of the present disclosure, the antibodies or antigen binding fragments thereof, for use in a method of detecting an ADA against anti-Factor XI and/or anti-Factor XIa antibodies or antigen-binding fragments thereof, may have the heavy and light chain sequences of the Fabs described in Table 1. More specifically, the antibody or antigen binding fragments thereof may have the heavy and light sequence of Antibody 2 and Antibody 1.

In certain embodiments of the present disclosure, the antibody or antigen binding fragment thereof, for use in a method of detecting an ADA against anti-Factor XI and/or anti-Factor XIa antibodies or antigen-binding fragments thereof, that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by Kabat and described in Table 1. For example, in certain embodiments of the present disclosure the antibody or antigen binding fragment thereof, for use in a method of detecting an ADA against anti-Factor XI and/or anti-Factor XIa antibodies or antigen-binding fragments thereof, that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by Chothia and described in Table 1. In other embodiments, the antibody or antigen binding fragment thereof, for use in a method of detecting an ADA against anti-Factor XI and/or anti-Factor XIa antibodies or antigen-binding fragments thereof, that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by the Combined system and described in Table 1. In certain embodiments of the present disclosure the antibody or antigen binding fragment thereof, for use in a method of detecting an ADA against anti-Factor XI and/or anti-Factor XIa antibodies or antigen-binding fragments thereof, that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by IMGT and described in Table 1.

In certain embodiments for use in the methods described herein (e.g., thereof, for use in a method of detecting an ADA against anti-Factor XI and/or anti-Factor XIa antibodies or antigen-binding fragments thereof), the present disclosure includes an antibody that specifically binds to FXI and/or FXIa comprising a heavy chain variable region CDR1 of SEQ ID NO: 3; a heavy chain variable region CDR2 of SEQ ID NO: 4; a heavy chain variable region CDR3 of SEQ ID NO: 5; a light chain variable region CDR1 of SEQ ID NO: 13; a light chain variable region CDR2 of SEQ ID NO: 14; and a light chain variable region CDR3 of SEQ ID NO: 15.

In certain embodiments, the present disclosure includes an antibody that specifically binds to FXI and/or FXIa comprising a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35, for use in a method of detecting an ADA against anti-Factor XI and/or anti-Factor XIa antibodies or antigen-binding fragments thereof.

In certain embodiments, the present disclosure includes an antibody that specifically binds to FXI and/or FXIa comprising a heavy chain variable region CDR1 of SEQ ID NO: 6; a heavy chain variable region CDR2 of SEQ ID NO: 7; a heavy chain variable region CDR3 of SEQ ID NO: 8; a light chain variable region CDR1 of SEQ ID NO: 16; a light chain variable region CDR2 of SEQ ID NO: 17; and a light chain variable region CDR3 of SEQ ID NO: 18, for use in a method of detecting an ADA against anti-Factor XI and/or anti-Factor XIa antibodies or antigen-binding fragments thereof.

In certain embodiments, the present disclosure includes an antibody that specifically binds to FXI and/or FXIa comprising a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of SEQ ID NO: 37; and a light chain variable region CDR3 of SEQ ID NO: 38, for use in a method of detecting an ADA against anti-Factor XI and/or anti-Factor XIa antibodies or antigen-binding fragments thereof.

In certain embodiments, provided herein is an antibody that specifically binds to FXI and/or FXIa comprising a heavy chain variable region CDR1 of SEQ ID NO: 43; a heavy chain variable region CDR2 of SEQ ID NO: 44; a heavy chain variable region CDR3 of SEQ ID NO: a light chain variable region CDR1 of SEQ ID NO: 47; a light chain variable region CDR2 of SEQ ID NO: 37 and a light chain variable region CDR3 of SEQ ID NO: 15, for use in a method of detecting an ADA against anti-Factor XI and/or anti-Factor XIa antibodies or antigen-binding fragments thereof.

In certain embodiments, provided herein is an antibody that specifically binds to FXI and/or FXIa comprising a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14 and a light chain variable region CDR3 of SEQ ID NO: 15, for use in a method of detecting an ADA against anti-Factor XI and/or anti-Factor XIa antibodies or antigen-binding fragments thereof.

In certain embodiments, the present disclosure includes antibodies or antigen binding fragments that specifically bind to FXI and/or FXIa as described in Table 1, for use in a method of detecting an ADA against anti-Factor XI and/or anti-Factor XIa antibodies or antigen-binding fragments thereof. In a specific embodiment for use in the methods described herein, the antibody, or antigen binding fragment, that binds FXI and/or FXIa is Antibody 2 and Antibody 1, for use in a method of detecting an ADA against anti-Factor XI and/or anti-Factor XIa antibodies or antigen-binding fragments thereof.

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody.

A human antibody that is “the product of” or “derived from” a particular human germline immunoglobulin sequence may contain amino acid differences as compared to the germline sequence, due to, for example, naturally occurring somatic mutations or intentional introduction of site-directed mutations. However, in the VH or VL framework regions, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene.

Typically, a recombinant human antibody will display no more than 10 amino acid differences from the amino acid sequence encoded by the human germline immunoglobulin gene in the VH or VL framework regions. In certain cases, the human antibody may display no more than 5, or even no more than 4, 3, 2, or 1 amino acid difference from the amino acid sequence encoded by the germline immunoglobulin gene. Examples of human germline immunoglobulin genes include, but are not limited to the variable domain germline fragments described below, as well as DP47 and DPK9.

### Homologous Antibodies

In yet other embodiments for use in the methods described herein (e.g., a method of detecting an ADA against anti-Factor XI and/or anti-Factor XIa antibodies or antigen-binding fragments thereof), the present disclosure provides an antibody, or an antigen binding fragment thereof, comprising amino acid sequences that are homologous to the sequences described in Table 1 (e.g., SEQ ID NOs: 29, 31, 39, or 41), and the antibody binds to an FXI and/or FXIa protein (e.g., human, rabbit, baboon, and cynomolgus monkey FXIa), and retains the desired functional properties of those antibodies described in Table 1 such as Antibody 2 and Antibody 1. In certain embodiments, such homologous antibodies retain the CDR amino acid sequences described in Table 1 (e.g., Kabat CDRs, Chothia CDRs, IMGT CDRs, or Combined CDRs).

For example, in some embodiments the present disclosure provides an isolated antibody, or a functional antigen binding fragment thereof, comprising a heavy chain variable domain and a light chain variable domain, wherein the heavy chain variable domain comprises an amino acid sequence that is at least 80%, at least 90%, or at least 95% identical to an amino acid sequence selected from the group consisting of SEQ ID NOs: 9 and 29; the light chain variable domain comprises an amino acid sequence that is at least 80%, at least 90%, or at least 95% identical to an amino acid sequence selected from the group consisting of SEQ ID NOs: 19 and 39; and the antibody specifically binds to FXI and/or FXIa (e.g., human, rabbit, baboon, and cynomolgus monkey FXIa), for use in a method of detecting an ADA against anti-Factor XI and/or anti-Factor XIa antibodies or antigen-binding fragments thereof. In certain embodiments, an isolated antibody, or a functional antigen binding fragment thereof, comprises a heavy chain variable domain and a light chain variable domain, wherein the heavy chain variable domain comprises an amino acid sequence that is at least 80%, at least 90%, or at least 95% identical to the amino acid sequence of SEQ ID NO: 9; the light chain variable domain comprises an amino acid sequence that is at least 80%, at least 90%, or at least 95% identical to the amino acid sequence of SEQ ID NO: 19; and the antibody specifically binds to FXI and/or FXIa (e.g., human, rabbit, baboon, and cynomolgus monkey FXIa), for use in a method of detecting an ADA against anti-Factor XI and/or anti-Factor XIa antibodies or antigen-binding fragments thereof. In certain embodiments, an isolated antibody, or a functional antigen binding fragment thereof, comprises a heavy chain variable domain and a light chain variable domain, wherein the heavy chain variable domain comprises an amino acid sequence that is at least 80%, at least 90%, or at least 95% identical to the amino acid sequence of SEQ ID NO: 29; the light chain variable domain comprises an amino acid sequence that is at least 80%, at least 90%, or at least 95% identical to the amino acid sequence of SEQ ID NO: 39; and the antibody specifically binds to FXI and/or FXIa (e.g., human, rabbit, baboon, and cynomolgus monkey FXIa), for use in a method of detecting an ADA against anti-Factor XI and/or anti-Factor XIa antibodies or antigen-binding fragments thereof. In certain embodiments of the present disclosure the heavy and light chain sequences of an antibody for use in a method of detecting an ADA against anti-Factor XI and/or anti-Factor XIa antibodies or antigen-binding fragments thereof, comprise HCDR1, HCDR2, HCDR3, LCDR1, LCDR2, and LCDR3 sequences as defined by Kabat, for example SEQ ID NOs: 3, 4, 5, 13, 14, and 15, respectively. In certain embodiments of the present disclosure the heavy and light chain sequences of an antibody for use in a method of detecting an ADA against anti-Factor XI and/or anti-Factor XIa antibodies or antigen-binding fragments thereof, comprise HCDR1, HCDR2, HCDR3, LCDR1, LCDR2, and LCDR3 sequences as defined by Chothia, for example SEQ ID NOs: 6, 7, 8, 16, 17, and 18, respectively. In certain embodiments, the heavy and light chain sequences of an antibody for use in a method of detecting an ADA against anti-Factor XI and/or anti-Factor XIa antibodies or antigen-binding fragments thereof, comprise HCDR1, HCDR2, HCDR3, LCDR1, LCDR2, and LCDR3 sequences as defined by the Combined system, for example SEQ ID NOs: 46, 4, 5, 33, 14, and 15, respectively. In certain embodiments, the heavy and light chain sequences of an antibody for use in a method of detecting an ADA against anti-Factor XI and/or anti-Factor XIa antibodies or antigen-binding fragments thereof, comprise HCDR1, HCDR2, HCDR3, LCDR1, LCDR2, and LCDR3 sequences as defined by IMGT, for example SEQ ID NOs: 43, 44, 45, 47, 37, and 15, respectively.

In other embodiments for use in the methods described herein (e.g., a method of detecting an ADA against anti-Factor XI and/or anti-Factor XIa antibodies or antigen-binding fragments thereof), the VH and/or VL amino acid sequences of an anti-Factor XI and/or anti-Factor XIa antibody may be 50%, 60%, 70%, 80%, 90%, 95%, 96%, 97%, 98% or 99% identical to the sequences set forth in Table 1. In other embodiments, the VH and/or VL amino acid sequences may be identical except for an amino acid substitution in no more than 1, 2, 3, 4 or 5 amino acid positions. An antibody having VH and VL regions having high (e.g., 80% or greater) identity to the VH and VL regions of those described in Table 1 can be obtained by mutagenesis (e.g., site-directed or PCR-mediated mutagenesis) of nucleic acid molecules encoding SEQ ID NOs: 10 or 30 and SEQ ID NOs: 20 and 40, respectively, followed by testing of the encoded altered antibody for retained function using the functional assays described herein.

In other embodiments for use in the methods described herein (e.g., a method of detecting an ADA against anti-Factor XI and/or anti-Factor XIa antibodies or antigen-binding fragments thereof), the full length heavy chain and/or full length light chain amino acid sequences of an anti-Factor XI and/or anti-Factor XIa antibody may be 50% 60%, 70%, 80%, 90%, 95%, 96%, 97%, 98% or 99% identical to the sequences set forth in Table 1 (e.g., SEQ ID NOs: 11 and/or 21, or 31 and/or 41). An antibody having a full length heavy chain and full length light chain having high (e.g., 80% or greater) identity to the full length heavy chains of any of SEQ ID NOs: 11 or 31, and full length light chains of any of SEQ ID NOs: 21 or 41, can be obtained by mutagenesis (e.g., site-directed or PCR-mediated mutagenesis) of nucleic acid molecules encoding such polypeptides, followed by testing of the encoded altered antibody for retained function using the functional assays described herein.

In one aspect, provided herein is an isolated antibody, or a functional antigen binding fragment thereof, for use in a method of detecting an ADA against anti-Factor XI and/or anti-Factor XIa antibodies or antigen-binding fragments thereof, comprising a heavy chain and a light chain, wherein the heavy chain comprises an amino acid sequence that is at least 80%, at least 90%, or at least 95% identical to an amino acid sequence selected from the group consisting of SEQ ID NOs: 11 and 31; the light chain comprises an amino acid sequence that is at least 80%, at least 90%, or at least 95% identical to an amino acid sequence selected from the group consisting of SEQ ID NOs: 21 and 41; and the antibody specifically binds to FXI and/or FXIa (e.g., human, rabbit, baboon, and cynomolgus monkey FXIa). In one embodiment, an isolated antibody, or a functional antigen binding fragment thereof, for use in a method of detecting an ADA against anti-Factor XI and/or anti-Factor XIa antibodies or antigen-binding fragments thereof, comprises a heavy chain and a light chain, wherein the heavy chain comprises an amino acid sequence that is at least 80%, at least 90%, or at least 95% identical to the amino acid sequence of SEQ ID NO: 11; the light chain comprises an amino acid sequence that is at least 80%, at least 90%, or at least 95% identical to the amino acid sequence of SEQ ID NO: 21; and the antibody specifically binds to FXI and/or FXIa (e.g., human, rabbit, baboon, and cynomolgus monkey FXIa). In certain embodiments, an isolated antibody, or a functional antigen binding fragment thereof, for use in a method of detecting an ADA against anti-Factor XI and/or anti-Factor XIa antibodies or antigen-binding fragments thereof, comprises a heavy chain and a light chain, wherein the heavy chain comprises an amino acid sequence that is at least 80%, at least 90%, or at least 95% identical to the amino acid sequence of SEQ ID NO: 31; the light chain comprises an amino acid sequence that is at least 80%, at least 90%, or at least 95% identical to the amino acid sequence of SEQ ID NO: 41; and the antibody specifically binds to FXI and/or FXIa (e.g., human, rabbit, baboon, and cynomolgus monkey FXIa). In certain aspects of the present disclosure the heavy and light chain sequences further comprise HCDR1, HCDR2, HCDR3, LCDR1, LCDR2, and LCDR3 sequences as defined by Kabat, for example SEQ ID NOs: 3, 4, 5, 13, 14, and 15, respectively. In certain embodiments of the present disclosure the heavy and light chain sequences of an antibody, or a functional antigen binding fragment thereof, for use in a method of detecting an ADA against anti-Factor XI and/or anti-Factor XIa antibodies or antigen-binding fragments thereof, comprise HCDR1, HCDR2, HCDR3, LCDR1, LCDR2, and LCDR3 sequences as defined by Chothia, for example SEQ ID NOs: 6, 7, 8, 16, 17, and 18, respectively. In certain embodiments, the heavy and light chain sequences of an antibody, or a functional antigen binding fragment thereof, for use in a method of detecting an ADA against anti-Factor XI and/or anti-Factor XIa antibodies or antigen-binding fragments thereof, comprise HCDR1, HCDR2, HCDR3, LCDR1, LCDR2, and LCDR3 sequences as defined by the Combined system, for example SEQ ID NOs: 46, 4, 5, 33, 14, and 15, respectively. In certain embodiments, the heavy and light chain sequences of an antibody, or a functional antigen binding fragment thereof, for use in a method of detecting an ADA against anti-Factor XI and/or anti-Factor XIa antibodies or antigen-binding fragments thereof, comprise HCDR1, HCDR2, HCDR3, LCDR1, LCDR2, and LCDR3 sequences as defined by IMGT, for example SEQ ID NOs: 43, 44, 45, 47, 37, and 15, respectively.

In other embodiments for use in the methods described herein, the full length heavy chain and/or full length light chain nucleotide sequences may be 60%, 70%, 80%, 90%, 95%, 96%, 97%, 98% or 99% identical to the sequences set forth in Table 1 (e.g., SEQ ID NOs: 12 and/or 22, or 32 and/or 42).

In other embodiments for use in the methods described herein, the variable regions of heavy chain and/or the variable regions of light chain nucleotide sequences may be 60%, 70%, 80%, 90%, 95%, 96%, 97%, 98% or 99% identical to the sequences set forth in Table 1 (e.g., SEQ ID NOs: 10 and/or 20, or 30 and/or 40).

As used herein, the percent identity between the two sequences is a function of the number of identical positions shared by the sequences (i.e., % identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The isolated anti-FXI and/or FXIa antibodies, or antigen binding fragments thereof, as described herein can be monoclonal antibodies, human or humanized antibodies, chimeric antibodies, single chain antibodies, Fab fragments, Fv fragments, F(ab′)2 fragments, or scFv fragments, and/or IgG isotypes (e.g., IgG1 such as human IgG1). In specific embodiments, anti-FXI and/or anti-FXIa antibodies described herein are recombinant human antibodies. In specific embodiments, anti-FXI and/or anti-FXIa antibodies described herein are human IgG1/lambda (λ) antibodies. In specific embodiments, anti-FXI and/or anti-FXIa antibodies described herein are human IgG1/lambda (λ) antibodies comprising an Fc domain engineered to reduce the potential for effector function (e.g., ADCC and/or CDC), for example a human Fc domain comprising D265A and/or P329A substitutions.

Additionally or alternatively, the protein sequences of the present disclosure can further be used as a “query sequence” to perform a search against public databases to, for example, identify related sequences. For example, such searches can be performed using the BLAST program (version 2.0) of Altschul, et al., 1990 J. Mol. Biol. 215:403-10.

Antibodies with Conservative Modifications

In certain other embodiments, an antibody of the present disclosure for use in the methods described herein (e.g., a method of detecting an ADA against anti-Factor XI and/or anti-Factor XIa antibodies or antigen-binding fragments thereof) has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibodies retain the desired functional properties of the FXIa-binding antibodies of the present disclosure.

Accordingly, for use in the methods described herein, in some embodiments the present disclosure provides an isolated antibody, or an antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of SEQ ID NOs: 3 and 23, and conservative modifications thereof; the heavy chain variable region CDR2 amino acid sequences are selected from the group consisting of SEQ ID NOs: 4 and 24, and conservative modifications thereof; the heavy chain variable region CDR3 amino acid sequences are selected from the group consisting of SEQ ID NOs: 5 and 25, and conservative modifications thereof; the light chain variable regions CDR1 amino acid sequences are selected from the group consisting of SEQ ID NOs: 13 and 33, and conservative modifications thereof; the light chain variable regions CDR2 amino acid sequences are selected from the group consisting of SEQ ID NOs: 14 and 34, and conservative modifications thereof; the light chain variable regions of CDR3 amino acid sequences are selected from the group consisting of SEQ ID NOs: 15 and 35, and conservative modifications thereof; and the antibody or antigen binding fragments thereof specifically binds to FXIa.

In one aspect, provided herein is an isolated antibody, or an antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of those described in Table 1, and conservative modifications thereof; the heavy chain variable region CDR2 amino acid sequences are selected from the group consisting of those described in Table 1, and conservative modifications thereof; the heavy chain variable region CDR3 amino acid sequences are selected from the group consisting of those described in Table 1, and conservative modifications thereof; the light chain variable regions CDR1 amino acid sequences are selected from the group consisting of those described in Table 1, and conservative modifications thereof; the light chain variable regions CDR2 amino acid sequences are selected from the group consisting of those described in Table 1, and conservative modifications thereof; the light chain variable regions of CDR3 amino acid sequences are selected from the group consisting of those described in Table 1, and conservative modifications thereof; and the antibody or antigen binding fragments thereof specifically binds to FXIa.

In other embodiments for use in the methods described herein, the antibody of the present disclosure is optimized for expression in a mammalian cell has a full length heavy chain sequence and a full length light chain sequence, wherein one or more of these sequences have specified amino acid sequences based on the antibodies described herein or conservative modifications thereof, and wherein the antibodies retain the desired functional properties of the FXIa binding antibodies of the present disclosure. Accordingly, the present disclosure provides an isolated antibody optimized for expression in a mammalian cell consisting of a full length heavy chain and a full length light chain wherein the full length heavy chain has amino acid sequences selected from the group of SEQ ID NOs: 11 or 31, and conservative modifications thereof; and the full length light chain has amino acid sequences selected from the group of SEQ ID NOs: 21 or 41, and conservative modifications thereof; and the antibody specifically binds to FXI and/or FXIa (e.g., human, rabbit, baboon, and cynomolgus monkey FXIa).

Antibodies that Bind to the Same Epitope

In some embodiments, the present disclosure provides antibodies that compete for the same epitope as the FXI and/or FXIa binding antibodies described in Table 1, for use in the methods described herein (e.g., a method of detecting an ADA against anti-Factor XI and/or anti-Factor XIa antibodies or antigen-binding fragments thereof). Additional antibodies can therefore be identified based on their ability to compete (e.g., to competitively inhibit the binding of, in a statistically significant manner, by binding to the same or overlapping epitope) with other antibodies of the present disclosure in FXI and/or FXIa binding assays. The ability of a test antibody to inhibit the binding of antibodies of the present disclosure to a FXI and/or FXIa protein demonstrates that the test antibody can compete with that antibody for binding to FXI and/or FXIa; such an antibody may, according to non-limiting theory, bind to the same or a related (e.g., a structurally similar or spatially proximal) epitope on the FXI and/or FXIa protein as the antibody with which it competes. In a certain embodiment, the antibody that binds to the same epitope on FXI and/or FXIa as the antibodies of the present disclosure is a human monoclonal antibody. Such human monoclonal antibodies can be prepared and isolated as described herein.

As used herein, an antibody “competes” for binding when the competing antibody binds to the same FXI and/or FXIa epitope as an antibody or antigen binding fragment of the present disclosure (e.g., Antibody 1 or Antibody 2) and inhibits FXI and/or FXIa binding of an antibody or antigen binding fragment of the present disclosure by more than 50% (for example, 80%, 85%, 90%, 95%, 98% or 99%) in the presence of an equimolar concentration of competing antibody. This may be determined, for instance, in a competitive binding assay, by any of the methods well known to those of skill in the art.

As used herein, an antibody or antigen binding fragment thereof does not “compete” with an FXI and/or FXIa antibody or antigen binding fragment of the present disclosure (e.g., Antibody 1 or Antibody 2) unless said competing antibody or antigen binding fragment thereof binds the same FXI and/or FXIa epitope, or an overlapping FXI and/or FXIa epitope, as an antibody or antigen binding fragment of the present disclosure. As used herein, a competing antibody or antigen binding fragment thereof does not include one which (i) sterically blocks an antibody or antigen binding fragment of the present disclosure from binding its target (e.g., if said competing antibody binds to a nearby, non-overlapping FXI and/or FXIa epitope and physically prevents an antibody or antigen binding fragment of the present disclosure from binding its target); and/or (ii) binds to a different, non-overlapping FXI and/or FXIa epitope and induces a conformational change to the FXI and/or FXIa protein such that said protein can no longer be bound by an FXI and/or FXIa antibody or antigen binding fragment of the present disclosure in a way that would occur absent said conformational change.

### Engineered and Modified Antibodies

In some embodiments, an antibody of the present disclosure, for use in the methods described herein (e.g., a method of detecting an ADA against anti-Factor XI and/or anti-Factor XIa antibodies or antigen-binding fragments thereof), further can be prepared using an antibody having one or more of the VH and/or VL sequences shown herein as starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998 Nature 332:323-327; Jones, P. et al., 1986 Nature 321:522-525; Queen, C. et al., 1989 Proc. Natl. Acad., U.S.A. 86:10029-10033; U.S. Pat. No. 5,225,539 to Winter, and U.S. Pat. Nos. 5,530,101; 5,585,089; 5,693,762 and 6,180,370 to Queen et al.)

Accordingly, another embodiment of the present disclosure pertains to an isolated antibody, or an antigen binding fragment thereof for use in a method of detecting an ADA against anti-Factor XI and/or anti-Factor XIa antibodies or antigen-binding fragments thereof, comprising a heavy chain variable region comprising CDR1 sequences having an amino acid sequence selected from the group consisting of SEQ ID NOs: 3 and 23; CDR2 sequences having an amino acid sequence selected from the group consisting of SEQ ID NOs: 4 and 24; CDR3 sequences having an amino acid sequence selected from the group consisting of SEQ ID NOs: 5 and 25, respectively; and a light chain variable region having CDR1 sequences having an amino acid sequence selected from the group consisting of SEQ ID NOs: 13 and 33; CDR2 sequences having an amino acid sequence selected from the group consisting of SEQ ID NOs: 14 and 34; and CDR3 sequences consisting of an amino acid sequence selected from the group consisting of SEQ ID NOs: 15 and 35, respectively. Thus, such antibodies contain the VH and VL CDR sequences of monoclonal antibodies, yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database, as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. Mol. Biol. 227:776-798; and Cox, J. P. L. et al., 1994 Eur. J Immunol. 24:827-836; the contents of each of which are expressly incorporated herein by reference.

An example of framework sequences for use in the antibodies of the present disclosure are those that are structurally similar to the framework sequences used by selected antibodies of the present disclosure, e.g., consensus sequences and/or framework sequences used by monoclonal antibodies of the present disclosure. The VH CDR1, 2 and 3 sequences, and the VL CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al). Frameworks that can be utilized as scaffolds on which to build the antibodies and antigen binding fragments described herein include, but are not limited to VH1A, VH1B, VH3, Vk1, Vl2, and Vk2.

Accordingly, for use in the methods described herein (e.g., a method of detecting an ADA against anti-Factor XI and/or anti-Factor XIa antibodies or antigen-binding fragments thereof), another embodiment of the present disclosure relates to isolated FXIa binding antibodies, or antigen binding fragments thereof, comprising a heavy chain variable region comprising an amino acid sequence selected from the group consisting of SEQ ID NOs: 9 and 29, or an amino acid sequence having one, two, three, four or five amino acid substitutions, deletions or additions in the framework region of such sequences, and further comprising a light chain variable region having an amino acid sequence selected from the group consisting of SEQ ID NOs: 19 or 39, or an amino acid sequence having one, two, three, four or five amino acid substitutions, deletions or additions in the framework region of such sequences.

Another type of variable region modification is mutation of amino acid residues within the VH and/or VL CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples Section. Conservative modifications (as discussed above) can be introduced. The mutations may be amino acid substitutions, additions or deletions. Moreover, typically no more than one, two, three, four or five residues within a CDR region are altered.

Accordingly, in another embodiment for use in the methods described herein (e.g., a method of detecting an ADA against anti-Factor XI and/or anti-Factor XIa antibodies or antigen-binding fragments thereof), the present disclosure provides isolated FXIa-binding antibodies, or antigen binding fragments thereof, consisting of a heavy chain variable region having a VH CDR1 region consisting of an amino acid sequence selected from the group having SEQ ID NOs: 3 and 23 or an amino acid sequence having one, two, three, four or five amino acid substitutions, deletions or additions as compared to SEQ ID NOs: 3 and 23; a VH CDR2 region having an amino acid sequence selected from the group consisting of SEQ ID NOs: 4 and 24 or an amino acid sequence having one, two, three, four or five amino acid substitutions, deletions or additions as compared to SEQ ID NOs: 4 and 24; a VH CDR3 region having an amino acid sequence selected from the group consisting of SEQ ID NOs: 5 and 25, or an amino acid sequence having one, two, three, four or five amino acid substitutions, deletions or additions as compared to SEQ ID NOs: 5 and 25; a VL CDR1 region having an amino acid sequence selected from the group consisting of SEQ ID NOs: 13 and 33, or an amino acid sequence having one, two, three, four or five amino acid substitutions, deletions or additions as compared to SEQ ID NOs: 13 and 33; a VL CDR2 region having an amino acid sequence selected from the group consisting of SEQ ID NOs: 14 and 34, or an amino acid sequence having one, two, three, four or five amino acid substitutions, deletions or additions as compared to SEQ ID NOs: 14 and 34; and a VL CDR3 region having an amino acid sequence selected from the group consisting of SEQ ID NOs: 15 and 35, or an amino acid sequence having one, two, three, four or five amino acid substitutions, deletions or additions as compared to SEQ ID NOs: 15 and 35.

Accordingly, in another embodiment for use in the methods described herein (e.g., a method of detecting an ADA against anti-Factor XI and/or anti-Factor XIa antibodies or antigen-binding fragments thereof), the present disclosure provides isolated FXIa-binding antibodies, or antigen binding fragments thereof, consisting of a heavy chain variable region having a VH CDR1 region consisting of an amino acid sequence selected from the group having SEQ ID NOs: 6 and 26 or an amino acid sequence having one, two, three, four or five amino acid substitutions, deletions or additions as compared to SEQ ID NOs: 6 and 26; a VH CDR2 region having an amino acid sequence selected from the group consisting of SEQ ID NOs: 7 and 27 or an amino acid sequence having one, two, three, four or five amino acid substitutions, deletions or additions as compared to SEQ ID NOs: 7 and 27; a VH CDR3 region having an amino acid sequence selected from the group consisting of SEQ ID NOs: 8 and 28, or an amino acid sequence having one, two, three, four or five amino acid substitutions, deletions or additions as compared to SEQ ID NOs: 8 and 28; a VL CDR1 region having an amino acid sequence selected from the group consisting of SEQ ID NOs: 16 and 36, or an amino acid sequence having one, two, three, four or five amino acid substitutions, deletions or additions as compared to SEQ ID NOs: 16 and 36; a VL CDR2 region having an amino acid sequence selected from the group consisting of SEQ ID NOs: 17 and 37, or an amino acid sequence having one, two, three, four or five amino acid substitutions, deletions or additions as compared to SEQ ID NOs: 17 and 37; and a VL CDR3 region having an amino acid sequence selected from the group consisting of SEQ ID NOs: 18 and 38, or an amino acid sequence having one, two, three, four or five amino acid substitutions, deletions or additions as compared to SEQ ID NOs: 18 and 38.

Antibodies with Extended Half Life

In some embodiments, the present disclosure provides for antibodies that specifically bind to FXIa protein which have an extended half-life in vivo, for use in the methods described herein (e.g., a method of detecting an ADA against anti-Factor XI and/or anti-Factor XIa antibodies or antigen-binding fragments thereof). An anti-Factor XI and/or anti-Factor XIa antibody having extended half-life may be relevant in the method of detecting an ADA, when the anti-Factor XI and/or anti-Factor XIa antibody is used in a method of treating a patient, and, therefore, physiological and clinical correlation to the ADA method and the therapeutic anti-Factor XI and/or anti-Factor XIa antibody is beneficial for treatment or maintenance of the disease or disorder of the patient.

Many factors may affect a protein's half-life in vivo. For examples, kidney filtration, metabolism in the liver, degradation by proteolytic enzymes (proteases), and immunogenic responses (e.g., protein neutralization by antibodies and uptake by macrophages and dendritic cells). A variety of strategies can be used to extend the half-life of the antibodies of the present disclosure. For example, by chemical linkage to polyethyleneglycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DARPins, avimers, affibodies, and anticalins; by genetic fusion to rPEG, albumin, domain of albumin, albumin-binding proteins, and Fc; or by incorporation into nanocarriers, slow release formulations, or medical devices.

To prolong the serum circulation of antibodies in vivo, inert polymer molecules such as high molecular weight PEG can be attached to the antibodies or a fragment thereof with or without a multifunctional linker either through site-specific conjugation of the PEG to the N- or C-terminus of the antibodies or via epsilon-amino groups present on lysine residues. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein. Methods for pegylating proteins are known in the art and can be applied to the antibodies of the present disclosure. See for example, EP 0 154 316 by Nishimura et al. and EP 0 401 384 by Ishikawa et al.

Other modified pegylation technologies include reconstituting chemically orthogonal directed engineering technology (ReCODE PEG), which incorporates chemically specified side chains into biosynthetic proteins via a reconstituted system that includes tRNA synthetase and tRNA. This technology enables incorporation of more than 30 new amino acids into biosynthetic proteins in E. coli, yeast, and mammalian cells. The tRNA incorporates a nonnative amino acid any place an amber codon is positioned, converting the amber from a stop codon to one that signals incorporation of the chemically specified amino acid.

Recombinant pegylation technology (rPEG) can also be used for serum halflife extension. This technology involves genetically fusing a 300-600 amino acid unstructured protein tail to an existing pharmaceutical protein. Because the apparent molecular weight of such an unstructured protein chain is about 15-fold larger than its actual molecular weight, the serum half-life of the protein is greatly increased. In contrast to traditional PEGylation, which requires chemical conjugation and repurification, the manufacturing process is greatly simplified and the product is homogeneous.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology includes the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

Antibodies having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; and U.S. Pat. No. 6,277,375.

Further, antibodies can be conjugated to albumin (e.g., human serum albumin; HSA) in order to make the antibody or antibody fragment more stable in vivo or have a longer half-life in vivo. The techniques are well-known in the art, see, e.g., International Publication Nos. WO 93/15199, WO 93/15200, and WO 01/77137; and European Patent No. EP 413,622. In addition, in the context of a bispecific antibody as described above, the specificities of the antibody can be designed such that one binding domain of the antibody binds to FXIa while a second binding domain of the antibody binds to serum albumin, preferably HSA.

The strategies for increasing half-life are especially useful in nanobodies, fibronectin-based binders, and other antibodies or proteins for which increased in vivo half-life is desired.

### Antibody Conjugates

In some embodiments, the present disclosure provides antibodies or fragments thereof, for use in the methods described herein (e.g., a method of detecting an ADA against anti-Factor XI and/or anti-Factor XIa antibodies or antigen-binding fragments thereof), that specifically bind to a FXIa protein recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In particular, the present disclosure provides fusion proteins comprising an antigen-binding fragment of an antibody described herein (e.g., a Fab fragment, Fd fragment, Fv fragment, F(ab)2 fragment, a VH domain, a VH CDR, a VL domain or a VL CDR) and a heterologous protein, polypeptide, or peptide for use in a method of detecting an ADA against anti-Factor XI and/or anti-Factor XIa antibodies or antigen-binding fragments thereof. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the present disclosure or fragments thereof (e.g., antibodies or fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to a FXIa protein may be recombined with one or more components, motifs, sections, parts, domains, fragments, etc. of one or more heterologous molecules.

Moreover, the antibodies or fragments thereof can be fused to marker sequences, such as a peptide to facilitate purification. In certain embodiments, the marker amino acid sequence is a hexa-histidine peptide (SEQ ID NO: 48), such as the tag provided in a pQE vector (QIAGEN, Inc., 9259 Eton Avenue, Chatsworth, CA, 91311), among others, many of which are commercially available. As described in Gentz et al., 1989, Proc. Natl. Acad. Sci. USA 86:821-824, for instance, hexa-histidine (SEQ ID NO: 48) provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

In other embodiments, antibodies for detection of an ADA may be conjugated to a diagnostic or detectable agent. Such detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I), carbon (14C), sulfur (35S), tritium (3H), indium (1151n, 113In, 112In, and 111In), technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142 Pr, 105Rh, 97Ru, 68Ge, 57Co, 65Zn, 85Sr, 32P, 153Gd, 169Yb, 51Cr, 54Mn, 75Se, 113Sn, and 117Tin; and positron emitting metals using various positron emission tomographies, and nonradioactive paramagnetic metal ions. In certain embodiments, an antibody for detection of an ADA is comprised in a ruthenylated detector cocktail.

In some embodiments, the present disclosure further encompasses uses of anti-Factor XI and/or anti-Factor XIa antibodies or fragments thereof conjugated to a therapeutic moiety, for use in a method of detecting an ADA against anti-Factor XI and/or anti-Factor XIa antibodies or antigen-binding fragments thereof. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

Further, an antibody or fragment thereof may be conjugated to a therapeutic moiety or drug moiety that modifies a given biological response, and such a conjugated antibody may be used in a method of detecting an ADA against anti-Factor XI and/or anti-Factor XIa antibodies or antigen-binding fragments thereof. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, an anti-angiogenic agent; or a biological response modifier such as, for example, a lymphokine.

Moreover, an antibody can be conjugated to therapeutic moieties such as a radioactive metal ion, such as alph-emiters such as 213Bi or macrocyclic chelators useful for conjugating radiometal ions, including but not limited to, 131In, 131LU, 131Y, 131Ho, 131Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Anti-Drug Antibody (ADA) Detection and Measurement

The present inventors have developed a novel approach for qualitatively and/or quantitatively detecting anti-Factor XI/XIa ADAs from a sample, which is effective in reducing and eliminating the interference problems caused by the presence of the drug or target in ADA detection.

### Assaying for ADA

Accordingly, described herein is a method of detecting an anti-drug antibody (ADA) against an anti-Factor XI and/or anti-Factor XIa antibody or antigen-binding fragment thereof, wherein the method comprises: incubating a sample with an acid to dissociate anti-Factor XI and/or anti-Factor XIa antibody-antigen complexes and/or dissociate anti-Factor XI and/or anti-Factor XIa antibody-ADA complexes present in the sample to create an acid digest, incubating the acid digest on a plate coated with the anti-Factor XI and/or anti-Factor XIa antibody or antigen-binding fragment thereof, neutralizing the acid digest, and detecting the presence of the ADA using a ruthenylated detector cocktail. In certain embodiments, the anti-Factor XI and/or anti-Factor XIa antibody is Antibody 1.

In some embodiments, the sample is a sample from a subject, e.g., a human subject. In some embodiments, the sample is selected from the group consisting of blood, plasma, or serum obtained from a subject, e.g., a human subject. In some embodiments, the sample is not obtained from a subject. In some embodiments, the sample is an in vitro sample prepared for testing, e.g., a sample comprising drug, target, and ADA. In certain embodiments of the method, the method comprises an initial step of preparing the sample.

Suitable acids for dissociating anti-Factor XI and/or anti-Factor XIa antibody-antigen complexes and/or dissociate anti-Factor XI and/or anti-Factor XIa antibody-ADA complexes include, for example and without limitation, acetic acid, propionic acid, lactic acid, malic acid, tartaric acid, citric acid or phosphoric acid, or mixtures thereof. In some embodiments, the acid is selected from the group consisting of acetic acid, citric acid, phosphoric acid, and mixtures thereof. In certain embodiments, the acid is acetic acid. It is contemplated herein that the pH of a particular acid or combination of acids may be adjusted to a desired pH, e.g., using methods known in the art. The concentration of acid, e.g., acetic acid, may be about 50 mM, about 100 mM, about 150 mM, about 200 mM, about 250 mM, about 300 mM, about 350 mM, about 400 mM, or about 500 mM. In certain embodiments, the concentration of acid, e.g., acetic acid, is about 300 mM.

The sample may be incubated with an acid for dissociation for a desired length of time. For example, the sample may be incubated with the acid for about 5 minutes, about 10 minutes, about 15 minutes, about 30 minutes, about 45 minutes, or about 60 minutes. In certain embodiments, the sample is incubated with the acid for about 10 minutes. In certain embodiments, the sample is incubated with the acid at room temperature. It is contemplated herein that the temperature of said incubation may affect the necessary time for incubation, i.e., a sample incubated below room temperature would require a longer incubation time, and a sample incubated above room temperature would require a shorter incubation time.

The assay plate may be coated first with a molecule (e.g., a protein) to enhance affinity of the drug (e.g., Antibody 1) for the plate. In certain embodiments, the plate is coated with streptavidin, and the drug is biotinylated. In certain embodiments, the plate is coated with nickel, and the drug has a histidine tag (e.g., 6×-His). In certain embodiments, the plate is coated with an antibody to a small peptide tag, and the drug is modified (e.g., genetically or chemically) to express said tag (e.g., V5, Flag, Myc, HA, GST, GFP, etc.). Alternative techniques to enhance affinity for a drug/protein are known in the art.

The concentration of the anti-Factor XI and/or anti-Factor XIa antibody or antigen-binding fragment thereof may vary depending on assay conditions. In some embodiments, the concentration of the anti-Factor XI and/or anti-Factor XIa antibody or antigen-binding fragment thereof (e.g., Antibody 1) is between about 0.05 μg/ml and about 0.45 μg/ml, between about 0.10 μg/ml and about 0.40 μg/ml, between about 0.15 μg/ml and about 0.35 μg/ml, or between about 0.20 μg/ml and about 0.30 μg/ml. In certain embodiments, the concentration of the anti-Factor XI and/or anti-Factor XIa antibody or antigen-binding fragment thereof (e.g., Antibody 1) is selected from the group consisting of 0.1 μg/ml, 0.25 μg/ml, 0.5 μg/ml, 0.75 μg/ml, and 1 μg/ml. In certain embodiments, the concentration of the anti-Factor XI and/or anti-Factor XIa antibody or antigen-binding fragment thereof (e.g., Antibody 1) is about 0.25 μg/ml.

In some embodiments, the method comprises a neturalizing step, wherein the neutralizing allows for the ADA to bind the anti-Factor XI and/or anti-Factor XIa antibody or antigen-binding fragment thereof (e.g., Antibody 1) on the coated plate. In certain embodiments, the neutralizing uses a base at a pH of about 8.0. It is contemplated herein that the pH of a particular base or combination of bases may be adjusted to a desired pH for neutralization, e.g., using methods known in the art.

Suitable bases for the neutralization step include, for example and without limitation, Tris, Phosphate, CAPS, CHAPS, EDTA, EGTA, HEPES, PIPES, MOPS, tricine, glycine, histidine, triethanolamine, and mixtures thereof. In certain embodiments, the base is selected from the group consisting of Tris, Phosphate, HEPES, triethanolamine, and mixtures thereof. In certain embodiments, the base is Tris. In certain embodiments, the base is Tris at a pH of about 8.0.

In some embodiments, the ADA are detected using a ruthenylated detector cocktail. In certain embodiments, the ruthenylated detector cocktail comprises an antibody. In certain embodiments, the antibody is an anti-human IgG, an anti-human IgM, an anti-human IgE, an anti-rabbit Ig, or any combination thereof. In certain embodiments, the ruthenylated detector cocktail comprises a ruthenylated anti-human IgG. In certain embodiments, the ruthenylated detector cocktail comprises a ruthenylated anti-human IgM. In certain embodiments, the ruthenylated detector cocktail comprises a ruthenylated anti-human IgE. In certain embodiments, the ruthenylated detector cocktail comprises a ruthenylated anti-rabbit Ig. In certain embodiments, the ruthenylated detector cocktail comprises a combination of said ruthenylated antibodies.

In some embodiments, the method comprises a wash step after any of the above steps. In certain embodiments, the method comprises a washing after the incubating. The washing step may be performed with a suitable wash buffer, e.g., Tris-HCl Buffered Saline (TBS), TBS Tween 20, phosphate-buffered saline (PBS), PBS Tween 20, etc. In some embodiments, the washing step is performed with PBS Tween 20. In certain embodiments, the washing step is performed with PBS 0.05% Tween 20.

In some embodiments, the plate is blocked prior to addition of the sample. Exemplary blocking buffers may comprise, for example and without limitation, bovine serum albumin (BSA), milk, goat serum, fetal bovine serum (FBS), horse serum (HS), or casein. In certain embodiments, the blocking buffer comprises BSA in phosphate buffered saline with tween-20 (PBS-T). In certain embodiments, the BSA in PBS-T is at a concentration of about 1%, about 2.5%, about 5%, about 7.5%, and about 10%. In certain embodiments, the BSA in PBS-T is at a concentration of about 5%.

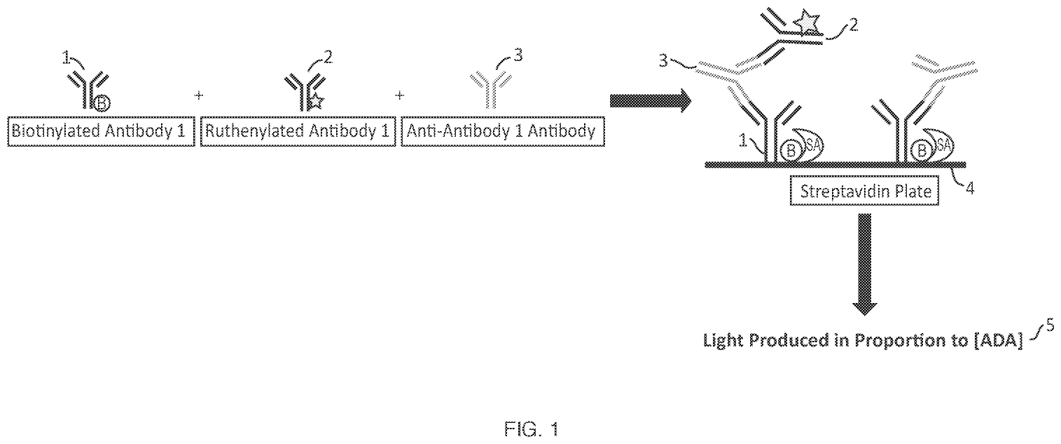

As shown in FIG. 1, in some embodiments of an assay described herein, a cocktail comprising a biotinylated Antibody 1 1, a ruthenylated Antibody 1 2, and an Anti-Antibody 1 Antibody 3 is incubated with a sample (e.g., blood, plasma, or serum) and added to a streptavidin-coated plate 4. In certain embodiments, a detectable signal (e.g., a chemiluminescent signal, e.g., light 5) is produced in proportion to the concentration of Anti-Antibody 1 antibodies. In certain embodiments, the sample (e.g., blood, plasma, or serum) is from a cynomolgus monkey.

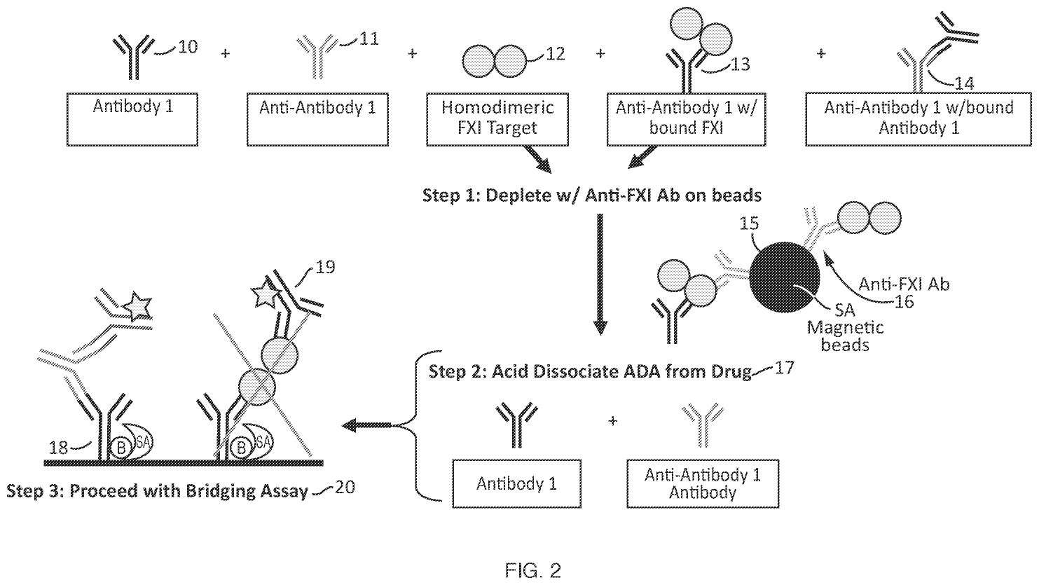

As shown in FIG. 2, in some embodiments of an assay described herein, a sample (e.g., blood, plasma, or serum) comprising Antibody 1 10, Anti-Antibody 1 11, homodimeric FXI and/or FXIa 12, Anti-Antibody 1-FXI/FXIa complexes 13, and Anti-Antibody 1-Antibody 1 complexes 14, is incubated with beads (e.g., streptavidin beads) 15, wherein the beads are coupled to anti-FXI antibodies 16, to form a depleted sample. The depleted sample is then incubated with an acid 17 to dissociate Antibody 1-Anti-Antibody 1 complexes present in the depleted sample, to form a dissociated sample. The dissociated sample is incubated with a cocktail comprising a biotinylated Antibody 1 18, a ruthenylated Antibody 1 19, and an Anti-Antibody 1 Antibody and added to a streptavidin-coated plate 20. In certain embodiments, a detectable signal (e.g., a chemiluminescent signal, e.g., light) is produced in proportion to the concentration of Anti-Antibody 1 antibodies. In certain embodiments, the sample (e.g., blood, plasma, or serum) is from a cynomolgus monkey.

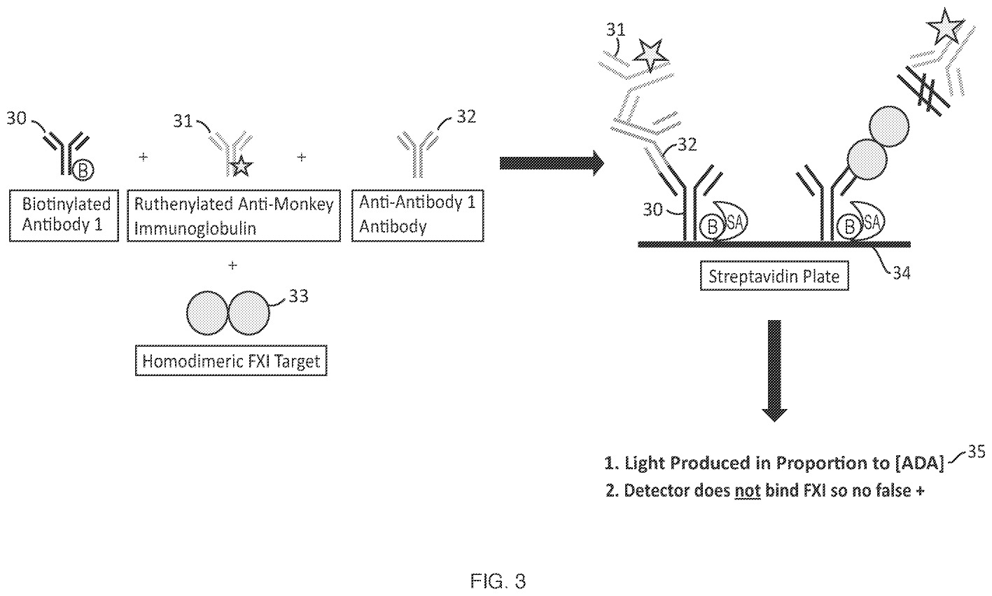

As shown in FIG. 3, in some embodiments of an assay described herein, a cocktail comprising a biotinylated Antibody 1 30, a ruthenylated Anti-Monkey Immunoglobulin 31, and an Anti-Antibody 1 Antibody 32 is incubated with a sample (e.g., blood, plasma, or serum) comprising homodimeric FXI and/or FXIa 33 and added to a streptavidin-coated plate 34. In certain embodiments, a detectable signal (e.g., a chemiluminescent signal, e.g., light 35) is produced in proportion to the concentration of Anti-Antibody 1 antibodies. In certain embodiments, the ruthenylated Anti-Monkey Immunoglobulin does not detect endogenous FXI/FXIa dimers. In certain embodiments, the sample (e.g., blood, plasma, or serum) is from a cynomolgus monkey.

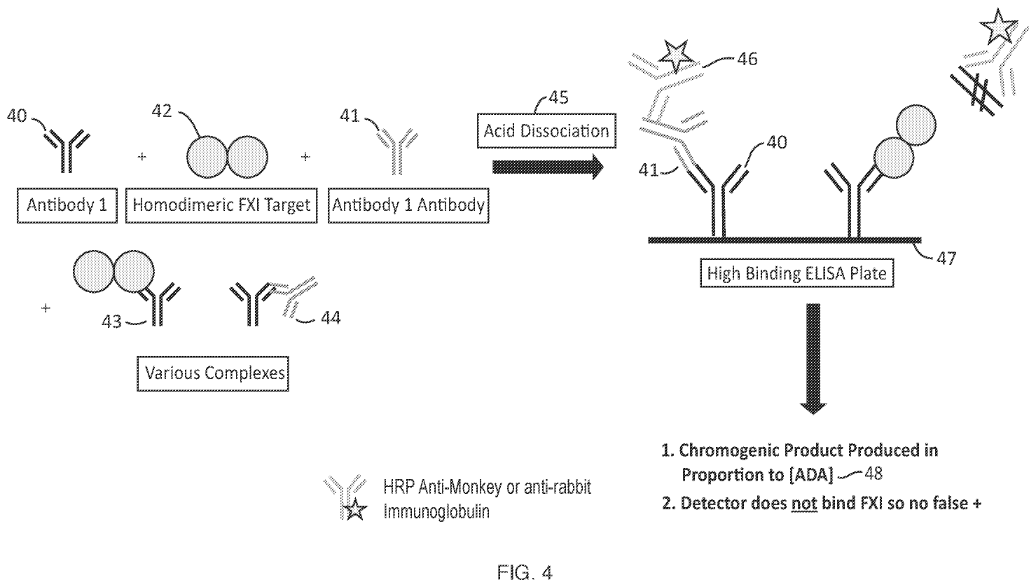

As shown in FIG. 4, in some embodiments of an assay described herein, a sample (e.g., blood, plasma, or serum) comprising Antibody 1 40, Anti-Antibody 1 41, homodimeric FXI and/or FXIa 42, Antibody 1-FXI/FXIa complexes 43, and Anti-Antibody 1-Antibody 1 complexes 44, is incubated with an acid 45 to dissociate Antibody 1-Anti-Antibody 1 complexes present in the sample, to form an acid digest. The acid digest is incubated with a cocktail comprising a biotinylated Antibody 1, a ruthenylated Anti-Monkey or Anti-Rabbit Immunoglobulin 46, and, an Anti-Antibody 1 Antibody and added to a high binding plate (e.g., a high binding ELISA plate 47). In certain embodiments, a detectable signal (e.g., a chemiluminescent signal, e.g., light 48) is produced in proportion to the concentration of Anti-Antibody 1 antibodies. In certain embodiments, the ruthenylated Anti-Monkey or Anti-Rabbit Immunoglobulin does not detect endogenous FXI/FXIa dimers. In certain embodiments, the sample (e.g., blood, plasma, or serum) is from a cynomolgus monkey.

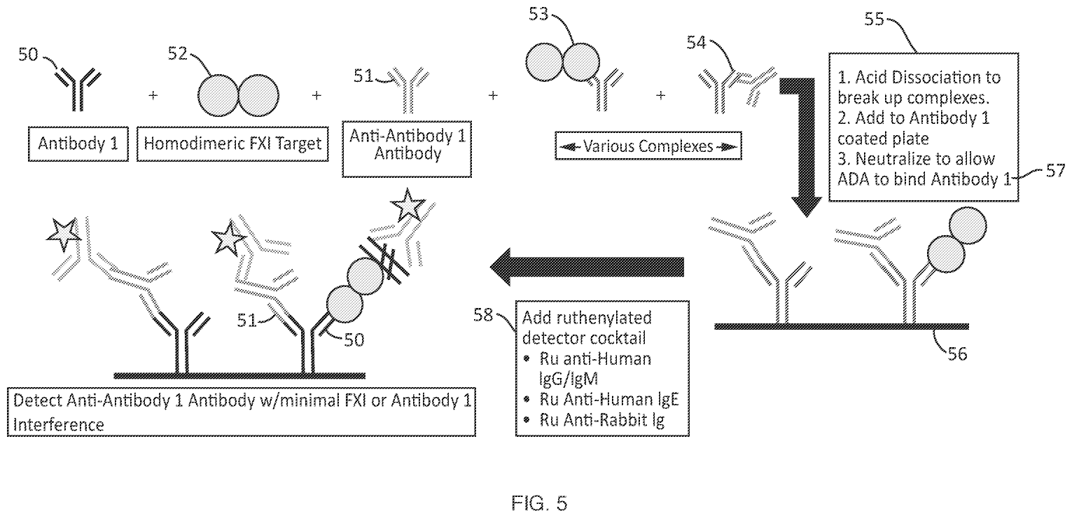

As shown in FIG. 5, in some embodiments of an assay described herein, a sample (e.g., blood, plasma, or serum) comprising Antibody 1 50, Anti-Antibody 1 51, homodimeric FXI and/or FXIa 52, Antibody 1-FXI/FXIa complexes 53, and Anti-Antibody 1-Antibody 1 complexes 54, is incubated with an acid 55 to dissociate Antibody 1-Anti-Antibody 1 complexes and/or Antibody 1-FXI/FXIa complexes present in the sample, to form an acid digest. The acid digest is added to a plate 56 coated with Antibody 1, and the acid digest is neutralized by addition of a suitable base 57 (e.g., Tris). A ruthenylated detector cocktail 58 comprising a ruthenylated anti-human IgG, anti-human IgM, ruthenylated anti-human IgE, and ruthenylated anti-Rabbit Ig. In certain embodiments, a detectable signal (e.g., a chemiluminescent signal, e.g., light) is produced in proportion to the concentration of Anti-Antibody 1 antibodies. In certain embodiments, the ruthenylated detector cocktail does not detect endogenous FXI/FXIa dimers. In certain embodiments, the ruthenylated detector cocktail does not detect Antibody 1. In certain embodiments, the sample (e.g., blood, plasma, or serum) is from a human.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Development of Factor XI and/or Factor XIa Anti-Drug Antibody Assay (ADA) for Cynomolgus Monkey Samples

Initially, the presence of anti-Factor XI and anti-Factor XIa (FXI/FXIa) anti-drug antibodies (ADA) in cynomolgus monkey serum were assayed using a standard bridging assay (FIG. 1). A cocktail comprising biotinylated Antibody 1, Ruthenylated Antibody 1, and an Antibody 1 ADA were added to a streptavidin coated plate. Light production from the chemiluminescent reaction was produced in proportion to the ADA. However, this approach resulted in a high percentage of false positives, as the endogenous protein target, homodimeric FXI/FXIa, was bridging the labeled Antibody 1.

It was therefore hypothesized that the introduction of a depletion step of endogenous homodimeric FXI/FXIa target would reduce the high false positive rate. Therefore, a further two steps: depletion of endogenous FXI/FXIa with anti-FXI/FXIa coated beads, followed by an acid dissociation of the drug from the antibody, were added prior to the bridging assay described above (FIG. 2). However, the depletion step with the anti-FXI antibody was inefficient, and no improvement in target interference in serum was detected.

Consequently, a new assay format, utilizing a Ruthenylated anti-monkey Ig as the detector, was tested (FIG. 3). Signal:Noise ratio determination revealed a significant improvement in FXI interference, as shown in Table 2 below.

TABLE 2
Signal-to-Noise Ratio for ADA assay with Ruthenylated anti-monkey Ig.

| | | Buffer | | | | Monkey Serum | | | | Human Serum | | | |
|---|---|---|---|---|---|---|---|---|---|---|---|---|---|
| | | | Target | Target | Target | | Target | Target | Target | | Target | Target | Target |
| | | | (1 | (10 | (50 | | (1 | (10 | (50 | | (1 | (10 | (50 |
| | ADA | | μg/ml) | μg/ml) | μg/ml) | | μg/ml) | μg/ml) | μg/ml) | | μg/ml) | μg/ml) | μg/ml) |
| | (ng/ | ADA | +ADA | +ADA | +ADA | ADA | +ADA | +ADA | +ADA | ADA | +ADA | +ADA | +ADA |
| | ml) | 1 | 2 | 3 | 4 | 5 | 6 | 7 | 8 | 9 | 10 | 11 | 12 |
| No | 500 | 58.44 | 64.23 | 57.07 | 54.52 | 51.11 | 50.68 | 43.01 | 36.84 | 44.79 | 46.47 | 38.99 | 35.10 |
| drug | 100 | 23.16 | 23.71 | 19.06 | 17.20 | 16.74 | 15.83 | 12.76 | 11.35 | 14.05 | 13.72 | 11.34 | 10.17 |
| Drug | 500 | 2.48 | 2.23 | 2.26 | 2.31 | 1.83 | 1.97 | 1.69 | 1.88 | 2.02 | 1.90 | 1.90 | 2.10 |
| (120 | | | | | | | | | | | | | |
| mg/ml) | 100 | 1.39 | 1.28 | 1.28 | 1.28 | 1.20 | 1.16 | 0.98 | 1.18 | 1.20 | 1.11 | 1.16 | 1.11 |

A schematic of the final assay format for cynomolgous ADA detection is depicted in FIG. 4. A summary of the assay validation parameters is shown in Table 3. Drug tolerance and target interference results are summarized in Table 4.

TABLE 3
Summary of ADA validation in cynomolgus monkey

| Validation | | Acceptance Criteria |
|---|---|---|
| Parameter | Result | met? |
| Screening | 47 individuals screened in | Report as |
| Cutpoint | 7 assays. Samples with | found. This is |
| (Tier 1) | S:N ratios > screening | a reasonable |
| | cutpoint of 1.16 are potential | cutpoint |
| | positive. | |
| Confirmatory | Antibody 1 at 400 μg/mL; | Report as |
| Cutpoint | 47 individuals in 7 | found. This is |
| (Tier 2) | assays. Confirmatory | a reasonable |
| | cutpoint was 29.35% | cutpoint |
| | inhibition. | |
| Titration | The highest dilution of | Report as |
| Cutpoint | sample that has S:N ratio | found. This is |
| (Tier 3) | >1.27 | a reasonable |
| | | cutpoint |
| Relative | 3.77 ng/mL in pooled | Yes. Meets FDA |
| Sensitivity | cynomolgus serum using | Guidance for |
| | rabbit polyclonal as surrogate | recommended |
| | | sensitivity |
| | | (500 ng/mL) |
| Precision | High Positive Control: 1.17-14.31% | Yes, all <20% |
| | Low Positive Control: 3.94-11.16% | |
| Selectivity | 10/10 non fortified individuals | Yes |
| | were negative | |
| | 10/10 fortified | |
| | individuals at LPC were positive | |

TABLE 4
Summary of ADA validation in cynomolgus monkey

| Drug Tolerance | | Target Interference | |
|---|---|---|---|
| Anti-Antibody 1 | Forecasted Drug | Anti-Antibody 1 | Forecasted Target |
| Antibody | Tolerance | Antibody | Interference |
| (ng/ml) | (μg/mL) | (ng/mL) | (ug/mL) |
| 500 | >500 | 500 | >199 |
| 100 | 69.5 | 100 | >199 |
| 33.3 | 11.7 | 33.3 | >199 |
| 11.1 | 6.92 | 11.1 | 71.0 |
| 3.70 | 2.65 | 3.70 | NA |

### Example 2: Development of Factor XI and/or Factor XIa Anti-Drug Antibody Assay (ADA) for Human Samples

The assay for cynomolgus monkey was adapted for validation in human serum. It was hypothesized that testing of Antibody 1 needed to be in the Fab format, to avoid nonspecific binding from Anti-human Ig. Antibody 1 was digested using pepsin or papain to generate F(ab′)₂ fragments. However, the resultant Fc fragment increased background to unacceptable levels. A negative purification to retain the Fc fragment produced very low yield of the F(ab′)₂ fragments and no reactivity. Accordingly, it was determined to use the full-length antibody format.

A schematic of the assay format is shown in FIG. 5. Briefly, a 96-well plate was coated with Antibody 1 at a concentration of 0.25 μg/ml, and subsequently blocked with 5% BSA in PBS-T. Samples were incubated in with 300 mM acetic acid for ten minutes to dissociate anti-Factor XI and/or anti-Factor XIa antibody-antigen complexes and to dissociate anti-Factor XI and/or anti-Factor XIa antibody-ADA complexes, forming an acid digest. The blocked plate was washed and 1 M of Tris at pH 8.0 was added for neutralization of the acid. The acid digest was then incubated on the plate coated with Antibody 1. A detector cocktail of anti-human IgG/IgM, ruthenylated anti-human IgE, and ruthenylated anti-rabbit Ig was added. The plate was washed after incubation with the acid digest. Chemilumiscence was then quantified to detect the Antibody 1 ADAs.

The FDA-Recommended Three Tier ADA Assay conditions were tested: cut point with 5% false positive rate in the Screening Assay, specificity of the response for the drug in the Confirmatory Assay, and a semi-quantitative estimate of ADA concentration in the Titer Assay.

The results of the Screening Assay are summarized in Table 5. The plate was coated at a concentration of 0.25 μg/ml. The ruthenylated detector cocktail anti-rabbit 0.1 μg/mL, anti-human IgG/M at 0.01 μg/mL, and anti-human IgE at 0.01 μg/mL. The plate was blocked using a buffer of 5% bovine serum albumin (BSA) in PBS-T. The assay results demonstrate high sensitivity with minimal background, FXI and drug interference.

TABLE 5
Results of Screening Assay for ADA in human samples.

| ADA | | | |
|---|---|---|---|
| Concentration | | ADA + FXI | ADA + Drug |
| (ng/ml) | ADA | (50 ug/ml) | (500 ug/ml) |
| 500 | 745.90 | 741.72 | 6.05 |
| 167 | 385.26 | 364.72 | 2.85 |
| 18.5 | 57.39 | 51.75 | 1.24 |
| 6.17 | 19.94 | 18.91 | 1.24 |
| 0 | 0.84 | 0.98 | 1.20 |

The results of the Confirmatory Assay are summarized in Table 6. As shown, sensitivity was established to at least 1.23 ng/mL.

TABLE 6
Results of Confirmatory Assay for ADA in human samples.

| ADA | Screening | | Confirmatory (400 μg/mL Antibody 1) | |
|---|---|---|---|---|
| Conc. | ECL | | ECL | % |
| (ng/mL) | Counts | S:N | Counts | Inhibition |
| 500 | 128742 | 521.22 | 828 | 99.36 |
| 100 | 37025 | 149.90 | 429 | 98.84 |
| 33.3 | 21558 | 87.28 | 307 | 98.58 |
| 11.1 | 7313 | 29.61 | 308 | 95.79 |
| 3.70 | 2545 | 10.30 | 327 | 87.15 |
| 1.23 | 939 | 3.80 | 266 | 71.67 |
| NC | 247 | 1.00 | 221 | 10.53 |

Results of drug tolerance titrations are summarized in Table 7. Values shown are signal:noise ratios.

TABLE 7
Results of Drug Tolerance Titrations for ADA in human samples.

| ADA | |
|---|---|
| Conc. | Drug Tolerance (μg/mL) |

| (ng/mL) | 0 | 12.5 | 25 | 50 | 100 | 500 |
|---|---|---|---|---|---|---|
| 500 | 264.67 | 20.38 | 13.60 | 9.48 | 6.12 | 2.91 |
| 100 | 93.31 | 5.41 | 3.97 | 2.84 | 2.31 | 1.40 |
| 33.3 | 37.43 | 2.60 | 2.04 | 1.70 | 1.55 | 1.09 |
| 11.1 | 14.45 | 1.56 | 1.45 | 1.28 | 1.17 | 1.24 |
| 3.70 | 5.89 | 1.22 | 1.15 | 1.19 | 1.25 | 1.27 |
| NC | 0.86 | 0.95 | 0.88 | 1.13 | 1.02 | 1.23 |

1-56. (canceled)

57. A method of detecting an anti-drug antibody (ADA) against an anti-Factor XI and/or anti-Factor XIa (anti-FXI/FXIa) antibody or antigen-binding fragment thereof, wherein the method comprises:
(a) incubating a sample with an acid to dissociate anti-FXI/FXIa antibody-antigen complexes and/or dissociate anti-FXI/FXIa antibody-ADA complexes present in the sample to create an acid digest;
(b) incubating the acid digest on a plate coated with the anti-Factor XI and/or anti-Factor XIa antibody or antigen-binding fragment thereof;
(c) neutralizing the acid digest; and
(d) detecting the presence of the ADA using a ruthenylated detector cocktail.

58. The method of claim 57, wherein the sample is selected from the group consisting of human blood, plasma, or serum.

59. The method of claim 57, wherein the method further comprises:
(a) an initial step of preparing the sample; and/or
(b) a washing step after the incubating.

60. The method of claim 57, wherein the acid is selected from the group consisting of acetic acid, citric acid, phosphoric acid, and mixtures thereof.

61. The method of claim 60, wherein the acid is acetic acid.

62. The method of claim 57, wherein neutralizing uses a base selected from the group consisting of Tris, Phosphate, HEPES, triethanolamine, and mixtures thereof.

63. The method of claim 62, wherein the base is Tris.

64. The method of claim 62, wherein:
(a) the base is at a pH of about 8.0; and/or
(b) the acid is at a concentration of about 300 mM.

65. The method of claim 57, wherein the ruthenylated detector cocktail comprises a detector antibody.

66. The method of claim 65, wherein the detector antibody is selected from the group consisting of an anti-human IgG, an anti-human IgM, an anti-human IgE, an anti-rabbit Ig, and any combination thereof.

67. The method of claim 57, wherein the plate is coated with streptavidin.

68. The method of claim 57, wherein the anti-FXI/FXIa antibody or antigen-binding fragment is coated on the plate in a buffer at a concentration selected from the group consisting of about 0.1 μg/mL, about 0.25 μg/mL, about 0.5 μg/mL, about 0.75 μg/mL, and about 1 μg/mL.

69. The method of claim 68, wherein the anti-anti-FXI/FXIa antibody or antigen-binding fragment is coated on the plate in a buffer at a concentration of about 0.25 μg/mL.

70. The method of claim 57, wherein the anti-FXI/FXIa antibody or antigen-binding fragment comprises:
i. a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34;and a light chain variable region CDR3 of SEQ ID NO: 35 ;
ii. a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of SEQ ID NO: 37;and a light chain variable region CDR3 of SEQ ID NO: 38;
iii. a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of SEQ ID NO: 37;and a light chain variable region CDR3 of SEQ ID NO: 15; or
iv. a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14;and a light chain variable region CDR3 of SEQ ID NO: 15.

71. The method of claim 57, wherein the anti-FXI/FXIa antibody or antigen-binding fragment comprises a heavy chain variable region (VH) comprising an amino acid sequence selected from the group consisting of SEQ ID NOs: 9, 29, and a VH with 90% identity thereto; and a light chain variable region (VL) comprising an amino acid sequence selected from the group consisting of SEQ ID NOs: 19, 39, and a VL with 90% identity thereto.

72. The method of claim 57, wherein the anti-FXI/FXIa antibody or antigen-binding fragment thereof comprises a VH comprising an amino acid sequence selected from the group consisting of SEQ ID NOs: 9 and 29; and a VL comprising an amino acid sequence selected from the group consisting of SEQ ID NOs: 19 and 39.

73. The method of claim 57, wherein the anti-FXI/FXIa antibody comprises a heavy chain comprising an amino acid sequence of SEQ ID NOs: 31, 11, or a heavy chain with 90% identity thereto; and a light chain comprising an amino acid sequence of SEQ ID NOs: 41, 21, or a light chain with 90% identity thereto.

74. The method of claim 57, wherein the anti-FXI/FXIa antibody comprises a heavy chain comprising an amino acid sequence of SEQ ID NO: 31 and a light chain comprising an amino acid sequence of SEQ ID NO: 41.

75. The method of claim 57, wherein the anti-FXI/FXIa antibody is a human monoclonal antibody.

76. The method of claim 75, wherein the anti-FXI/FXIa antibody is a human IgG1isotype.

77. The method of claim 76, wherein the anti-FXI/FXIa antibody comprises an antibody Fc domain comprising D265A and P329A substitutions.
